(19) United States
(12) Patent Application Publication
(10) Pub. No.: US 2012/0094277 A1
(43) Pub. Date: Apr. 19, 2012

(54) Methods of producing competitive aptamer FRET reagents and assays

(75) Inventors: John G. Bruno, San Antonio, TX (US); Joseph Chanpong, Chapel Hill, NC (US)

(73) Assignee: PRONUCLEOTEIN BIOTECHNOLOGIES, LLC

(21) Appl. No.: 13/373,993

(22) Filed: Dec. 7, 2011

Related U.S. Application Data
(63) Continuation of application No. 12/011,675, filed on Jan. 29, 2008, now abandoned.

(51) Int. Cl. C12Q 1/70; C12Q 1/68; G01N 33/53

(52) U.S. Cl. 435/5; 436/501; 435/6.15

(57) ABSTRACT

Methods are described for the production and use of fluorescence resonance energy transfer (FRET)-based competitive displacement aptamer assay formats. The assay schemes involve FRET in which the analyte (target) is quencher (Q)-labeled and previously bound by a fluorophore (F)-labeled aptamer such that when unlabeled analyte is added to the system and excited by specific wavelengths of light, the fluorescence intensity of the system changes in proportion to the amount of unlabeled analyte added. Alternatively, the aptamer can be Q-labeled and previously bound to an F-labeled analyte so that when unlabeled analyte enters the system, the fluorescence intensity also changes in proportion to the amount of unlabeled analyte. The F or Q is covalently linked to nucleotide triphosphates (NTPs), which are incorporated into the aptamer by various nucleic acid polymerases, such as Taq or Deep Vent Exo⁻ during PCR or asymmetric PCR, and then selected by affinity chromatography, size-exclusion, and fluorescence techniques.

### CROSS-REFERENCE TO RELATED APPLICATIONS

This application is a continuation of co-pending U.S. application Ser. No. 12/011,675 filed on Jan. 29, 2008, which is incorporated herein by reference.

### BACKGROUND OF THE INVENTION

1. Field of the Invention

The present invention relates to the field of aptamer- and nucleic acid-based diagnostics. More particularly, it relates to methods for the production and use of fluorescence resonance energy transfer (“FRET”) DNA or RNA aptamers for competitive displacement aptamer assay formats. The present invention provides for aptamer-related FRET assay schemes involving competitive displacement formats in which the aptamer contains fluorophores (“F”) (is F-labeled) and the target contains quenchers (“Q”) (is Q-labeled), or vice versa. The aptamer can be F-labeled or Q-labeled by incorporation of the F or Q derivatives of nucleotide triphosphates. Incorporation may be accomplished by simple chemical conjugations through bifunctional linkers, or key functional groups such as aldehydes, carbodiimides, carboxyls, N-hydroxy-succinimide (NHS) esters, thiols, etc.

2. Background Information

Competitive displacement aptamer FRET is a new class of assay desirable for its use in rapid (within minutes), one-step, homogeneous assays involving no wash steps (simple bind and detect quantitative assays). A fluorophore is a molecule (e.g., colored dye) which emits light at a specific range of wavelengths or segment of the spectrum after excitation by light of a lower wavelength or lower range of wavelengths versus the emission wavelengths. Different types of fluorophores emit energy at different wavelengths or spectral ranges. A quencher is a molecule which absorbs light energy (or photons) at a specific spectral range of wavelengths and does not re-emit light, but converts virtually all of the excitation light energy into invisible vibrations (e.g., infrared or heat). Different types of quenchers absorb energy at different wavelengths or spectral ranges. Others have described FRET-aptamer methods for various target analytes that consist of placing the F and Q moieties either on the 5′ and 3′ ends respectively to act like a “molecular (aptamer) beacon” or placing only F in the heart of the aptamer structure to be “quenched” by another proximal F or the DNA or RNA itself. These preceding FRET-aptamer methods are all highly engineered and based on some prior knowledge of particular aptamer sequences and secondary structures, thereby enabling clues as to where F might be placed in order to optimize FRET results.

### SUMMARY OF THE INVENTION

The nucleic acid-based “molecular beacons” snap open upon binding to an analyte or upon hybridizing to a complementary sequence, but beacons have always been end-labeled with F and Q at the 3′ and 5′ ends. The present invention provides that F-labeled or Q-labeled aptamers may be labeled anywhere in their structure that places the F or Q within the Förster distance of approximately 60-85 Angstroms of the corresponding F or Q on the labeled target analyte to achieve quenching prior to or after target analyte binding to the aptamer “binding pocket” (typically a “loop” in the secondary structure). In order to achieve FRET, the F and Q molecules used can include any number of appropriate fluorophores and quenchers as long as they are spectrally matched so the emission spectrum of F overlaps significantly (greater than 50%) with the absorption spectrum of Q, such that when the F and the Q are moved into or out of functional proximity (the Förster distance of less than or equal to 85 Angstroms), there is a detectable change in the fluorescent signal of the aptamer—either more detectable light when the Q is moved away from the F, or less detectable light when the Q is moved near the F.

A process in which F and Q are incorporated into an aptamer population is generally referred to as “doping.” The present invention provides a new method for natural selection of F-labeled or Q-labeled aptamers that contain F-NTPs or Q-NTPs in the heart of an aptamer binding loop or pocket by PCR, asymmetric PCR (using a 100:1 forward:reverse primer ratio), or other enzymatic means. The present invention describes a strain of aptamer in which F and Q are incorporated into an aptamer population via their nucleotide triphosphate derivatives (for example, Alexfluor™-NTP's, Cascade Blue®-NTP's, Chromatide®-NTP's, fluorescein-NTP's, rhodamine-NTP's, Rhodamine Green™-NTP's, tetramethylrhodamine-dNTP's, Oregon Green®-NTP's, and Texas Red®-NTP's may be used to provide the fluorophores, while dabcyl-NTP's, Black Hole Quencher or BHQ™-NTP's, and QSY™ dye-NTP's may be used for the quenchers) by PCR after several rounds of selection and amplification without the F- and Q-modified bases. The advantage of this F or Q “doping” method is two-fold: 1) the method allows nature to take its course and select the most sensitive F-labeled or Q-labeled aptamer target interactions in solution, and 2) the positions of F or Q within the aptamer structure can be determined via exonuclease digestion of the F-labeled or Q-labeled aptamer followed by mass spectral analysis of the resulting fragments, thereby eliminating the need to “engineer” the F or Q moieties into a prospective aptamer binding pocket or loop. Sequence and mass spectral data can be used to further optimize the competitive aptamer FRET assay performance after natural selection as well.

If the target molecule is a larger water-soluble molecule such as a protein, glycoprotein, or other water soluble macromolecule, then exposure of the nascent F-labeled and Q-labeled DNA or RNA random library to the free target analyte is done in solution. If the target is a soluble protein or other larger water-soluble molecule, then the optimal FRET-aptamer-target complexes are separated by size-exclusion chromatography. The FRET-aptamer-target complex population of molecules is the heaviest subset in solution and will emerge from a size-exclusion column first, followed by unbound FRET-aptamers and unbound proteins or other targets. Among the subset of analyte-bound aptamers there will be heterogeneity in the numbers of F- and Q-NTP's that are incorporated as well as nucleotide sequence differences, which will again effect the mass, electrical charge, and weak interaction capabilities (e.g., hydrophobicity and hydrophilicity) of each analyte-aptamer complex. These differences in physical properties of the aptamer-analyte complexes can then be used to separate out or partition the bound from unbound analyte-aptamer complexes.

If the target is a small molecule (generally defined as a molecule with molecular weight of ≦1,000 Daltons), then exposure of the nascent F-labeled and Q-labeled DNA or RNA random library to the target is done by immobilizing the target. The small molecule can be immobilized on a column, membrane, plastic or glass bead, magnetic bead, quantum dot, or other matrix. If no functional group is available on the small molecule for immobilization, the target can be immobilized by the Mannich reaction (formaldehyde-based condensation reaction) on a PharmaLink™ column from Pierce Chemical Co. Elution of bound DNA from the small molecule affinity column, membrane, beads or other matrix by use of 0.2-3.0M sodium acetate at a pH of between 3 and 7.

The candidate FRET-aptamers are separated based on physical properties such as charge or weak interactions by various types of HPLC, digested at each end with specific exonucleases (snake venom phosphodiesterase on the 3′ end and calf spleen phosphodiesterase on the 5′ end). The resulting oligonucleotide fragments, each one bases shorter than the predecessor, are subjected to mass spectral analysis which can reveal the nucleotide sequences as well as the positions of F and Q within the FRET-aptamers. Once the FRET-aptamer sequence is known with the positions of F and Q, it can be further manipulated during solid-phase DNA or RNA synthesis in an attempt to make the FRET assay more sensitive and specific.

The competitive displacement aptamer FRET assay format of the present invention is unique. The competitive format generally requires a lower affinity aptamer in order to be able to release the F-labeled or Q-labeled target analyte and allow competition for the binding site. This may lead to less sensitivity in some assays.

When running an assay, an aptamer is incorporated. In order to interact with the target molecule, the aptamer has a binding pocket or site. It is anticipated in some embodiments that the binding pocket is comprised of 3 to 6 nucleotides. These 3 or more nucleotides have a specific sequence or arrangement so as to confer the appropriate volume and conformation in 3-dimensional space to enable optimal binding to the target molecule. Where the target molecule can be any of the type described herein.

The described competitive FRET aptamer uses unique aptamer sequences. The following sequences are all aptamers that bind foodborne pathogens such as E. coli O157:H7, Salmonella typhimurium and a surface protein from Listeria monocytogenes called “Listeriolysin.” F=forward and R=reverse primed sequences. The invention described herein may use one or more of the following aptamer sequences (the following aptamer sequences are collectively referred to as the “SEQ Aptamers.”) (The SEQ Aptamer identifiers are arranged alphabetically by aptamer target, and are listed 5′ to 3′ from left to right):

### Acetylcholine (ACh) Aptamer Sequences:

ACh1a For
ATACGGGAGCCAACACCACGATACCCGCTTATGAATTTTAAATTAATT
GTGATCAGAGCAGGTGTGACGGAT
ACh1a Rev
ATCCGTCACACCTGCTCTGATCACAATTAATTTAAAATTCATAAGCGG
GTATCGTGGTGTTGGCTCCCGTAT
ACh 1b For
ATACGGGAGCCAACACCAACTTTCACACATACTTGTTATACCACACGA
TCTTTTAGAGCAGGTGTGACGGAT
ACh 1b Rev
ATCCGTCACACCTGCTCTAAAAGATCGTGTGGTATAACAAGTATGTGT
GAAAGTTGGTGTTGGCTCCCGTAT
ACh 2 For
ATACGGGAGCCAACACCACTTTGTAACTCATTTGTAGTTTGGGTTGCT
CCCCCTAGAGCAGGTGTGACGGAT
ACh 2 Rev
ATCCGTCACACCTGCTCTAGGGGGAGCAACCCAAACTACAAATGAGTT
ACAAAGTGGTGTTGGCTCCCGTAT
ACh 3 For
ATACGGGAGCCAACACCATTTCCCGCTTATCTTCATCCACTGCTTAGC
ATATGTAGAGCAGGTGTGACGGAT
ACh 3 Rev
ATCCGTCACACCTGCTCTACATATGCTAAGCAGTGGATGAAGATAAGC
GGGAAATGGTGTTGGCTCCCGTAT
ACh 5 For
ATACGGGAGCCAACACCAGGCACTGTATCACACCGTCAAGAATGTGAT
CCCCTGAGAGCAGGTGTGACGGAT
ACh 5 Rev
ATCCGTCACACCTGCTCTCAGGGGATCACATTCTTGACGGTGTGATAC
AGTGCCTGGTGTTGGCTCCCGTAT
ACh 6 For
ATACGGGAGCCAACACCATGTCATTTACCTTCATCATGACAGTGTTAG
TATACGAGAGCAGGTGTGACGGAT
ACh 6 Rev
ATCCGTCACACCTGCTCTAGGGGATCAAAGCTATGCGACCATGCGAGT
GGATACTGGTGTTGGCTCCCGTAT
ACh 7 For
ATACGGGAGCCAACACCAGTTGCCGCCTACCTTGATTATTCTACATTA
CCCGTTAGAGCAGGTGTGACGGAT
ACh 7 Rev
ATCCGTCACACCTGCTCTAACGGGTAATGTAGAATAATCAAGGTAGGC
GGCAACTGGTGTTGGCTCCCGTAT
ACh 8 For
ATACGGGAGCCAACACCAGTATACATACGAAGAGTTGAAACCAATGCT
TCGTTCAGAGCAGGTGTGACGGAT
ACh 8 Rev
ATCCGTCACACCTGCTCTGAACGAAGCATTGGTTTCAACTCTTCGTAT
GTATACTGGTGTTGGCTCCCGTAT
ACh 9 For
ATACGGGAGCCAACACCATACCCCGAATGGCTGTTTTCAGTACCAATA
TGACTCAGAGCAGGTGTGACGGAT
ACh 9 Rev
ATCCGTCACACCTGCTCTGAGTCATATTGGTACTGAAAACAGCCATTC
GGGGTATGGTGTTGGCTCCCGTAT
ACh 10 For
ATACGGGAGCCAACACCACTGTCACGATCGTCGTTCCTTTTAATCCTT
GTGTCTAGAGCAGGTGTGACGGAT
ACh 10 Rev
ATCCGTCACACCTGCTCTAGACACAAGGATTAAAAGGAACGACGATCG
TGACAGTGGTGTTGGCTCCCGTAT
ACh 11 For
ATACGGGAGCCAACACCACTGGACACTGACCCTCGCACTAGCTTTCTG
ACGGGTAGAGCAGGTGTGACGGAT
ACh 11 Rev
ATCCGTCACACCTGCTCTACCCGGCCGAAGAATAGTGCTCGGTACTTA
GTCGCGTGGTGTTGGCTCCCGTAT
ACh 12 For
ATACGGGAGCCAACACCATTTGGACTTTAAATAGTGGACTCCTTCTTT
GTCTCGAGAGCAGGTGTGACGGAT
ACh 12 Rev
ATCCGTCACACCTGCTCTCGAGACAAAGAAGGAGTCCACTATTTAAAG
TCCAAATGGTGTTGGCTCCCGTAT
A25 For
ATACGGGAGCCAACACCA-TCATTTGCAAATATGAATTCCACTTAAAG
AAATTCA-AGAGCAGGTGTGACGGAT
A25 Rev
ATCCGTCACACCTGCTCTTGAATTTCTTTAAGTGGAATTCATATTTGC
AAATGATGGTGTTGGCTCCCGTAT
Acyl Homoserine Lactone (AHL) Quorum Sensing
Molecules (N-Decanoyl-DL-Homoserine Lactone)
Dec AHL 1 For
ATACGGGAGCCAACACCATCCTAACTGGTCTAATTTTTGCTGTTACCG
ATCCCGAGAGCAGGTGTGACGGAT
Dec AHL 1 Rev
ATCCGTCACTCCTGCTCTCGGGATCGGTAACAGCAAAAATTAGACCAG
TTAGGATGGTGTTGGCTCCCGTAT
Dec AHL 13 For
ATACGGGAGCCAACACCAGCCTGACGAAAAAATTTTATCACTAAGTGA
TACGCAAGAGCAGGTGTGACGGAT
Dec AHL 13 Rev
ATCCGTCACACCTGCTCTTGCGTATCACTTAGTGATAAAATTTTTTCG
TCAGGCTGGTGTTGGCTCCCGTAT
Dec AHL 14 For
ATACGGGAGCCAACACCAGACCTACTTCAGAAACGGAAATGTTCTTAG
CCGTCAGAGCAGGTGTGACGGAT
Dec AHL 14 Rev
ATCCGTCACACCTGCTCTGACGGCTAAGAACATTTCCGTTTCTGAAGT
AGGTCTGGTGTTGGCTCCCGTAT
Dec AHL 15 For
ATACGGGAGCCAACACCAGGCCAACGAAACTCCTACTACATATAATGC
TTATGCAGAGCAGGTGTGACGGAT
Dec AHL 15 Rev
ATCCGTCACACCTGCTCTGCATAAGCATTATATGTAGTAGGAGTTTCG
TTGGCCTGGTGTTGGCTCCCGTAT
Dec AHL 17 For
ATACGGGAGCCAACACCATCCTAACTGGTCTAATTTTTGCTGTTACCG
ATCCCGAGAGCAGGTGTGACGGAT
Dec AHL 17 Rev
ATCCGTCACACCTGCTCTCGGGATCGGTAACAGCAAAAATTAGACCAG
TTAGGATGGTGTTGGCTCCCGTAT

### Bacillus Thurinjiensis Spore Aptamer Sequence:

CATCCGTCACACCTGCTCTGGCCACTAACATGGGGACCAGGTGGTGTT
GGCTCCCGTATC

### Botulinum Toxin (BoNT Type A) Aptamer Sequences:

BoNT A Holotoxin (Heavy Chain plus Light Chain
Linked Together)
CATCCGTCACACCTGCTCTGCTATCACATGCCTGCTGAAGTGGTGTTG
GCTCCCGTATCA
BoNT A 50 kd Enzymatic Light Chain
BoNT A Light Chain 1
CATCCGTCACACCTGCTCTGGGGATGTGTGGTGTTGGCTCCCGTATCA
AGGGCGAATTCT
BoNT A Light Chain 2
CATCCGTCACACCTGCTCTGATCAGGGAAGACGCCAACACGTGGTGTT
GGCTCCCGTATCA
BoNT A Light Chain 3
CATCCGTCACACCTGCTCTGGGTGGTGTTGGCTCCCGTATCAAGGGCG
AATTCTGCAGATA

### Campylobacter Jejuni Binding Aptamers:

C 1
CATCCGTCACACCTGCTCTGGGGAGGGTGGCGCCCGTCTCGGTGGTGT
TGGCTCCCGTATCA
C 2
CATCCGTCACACCTGCTCTGGGATAGGGTCTCGTGCTAGATGTGGTGT
TGGCTCCCGTATCA
C 3
CATCCGTCACACCTGCTCTGGACCGGCGCTTATTCCTGCTTGTGGTGT
TGGCTCCCGTATCA
C 4
CATCCGTCACACCTGCYCTGGAGCTGATATTGGATGGTCCGGTGGTGT
TGGCTCCCGTATCA
C 5
CATCCGTCACACCTGCYCYGCCCAGAGCAGGTGTGACGGATGTGGTGT
TGGCTCCCGTATCA
C 6
CATCCGTCACACCTGCYCYGCCGGACCATCCAATATCAGCTGTGGTGT
TGGCTCCCGTATCA
Diazinon Binding Aptamers
D12 Forward
ATACGGGAGCCAACACCATTAAATCAATTGTGCCGTGTTGGTCTTGTC
TCATCGAGAGCAGGTGTGACGGAT
D12 Reverse
ATCCGTCACACCTGCTCTCGATGAGACAAGACCAACACGGCACAATTG
ATTTAATGGTGTTGGCTCCCGTAT
D17 Forward
ATACGGGAGCCAACACCATTTTTATTATCGGTATGATCCTACGAGTTC
CTCCCAAGAGCAGGTGTGACGGAT
D17 Reverse
ATCCGTCACACCTGCTCTTGGGAGGAACTCGTAGGATCATACCGATAA
TAAAAATGGTGTTGGCTCCCGTAT
D18 Forward
ATACGGGAGCCAACACCACCGTATATCTTATTATGCACAGCATCACGA
AAGTGCAGAGCAGGTGTGACGGAT
D18 Reverse
ATCCGTCACACCTGCTCTGCACTTTCGTGATGCTGTGCATAATAAGAT
ATACGGTGGTGTTGGCTCCCGTAT
D19 Forward
ATACGGGAGCCAACACCATTAACGTTAAGCGGCCTCACTTCTTTTAAT
CCTTTCAGAGCAGGTGTGACGGAT
D19 Reverse
ATCCGTCACACCTGCTCTGAAAGGATTAAAAGAAGTGAGGCCGCTTAA
CGTTAATGGTGTTGGCTCCCGTAT
D20 Forward
ATCCGTCACACCTGCTCTAATATAGAGGTATTGCTCTTGGACAAGGTA
CAGGGATGGTGTTGGCTCCCGTAT
D20 Reverse
ATACGGGAGCCAACACCATCCCTGTACCTTGTCCAAGAGCAATACCTC
TATATTAGAGCAGGTGTGACGGAT
D25 Forward
ATACGGGAGCCAACACCATTAACGTTAAGCGGCCTCACTTCTTTTAAT
CCTTTCAGAGCAGGTGTGACGGAT
D25 Reverse
ATCCGTCACACCTGCTCTGAAAGGATTAAAAGAAGTGAGGCCGCTTAA
CGTTAATGGTGTTGGCTCCCGTAT

Glucosamine (from LPS) Forward Aptamer Sequences:

G 1 For
ATCCGTCACACCTGCTCTAATTAGGATACGGGGCAACAGAACGAGAGG
GGGGAATGGTGTTGGCTCCCGTAT
G 2 For
ATCCGTCACACCTGCTCTCGGACCAGGTCAGACAAGCACATCGGATAT
CCGGCTGGTGTTGGCTCCCGTAT
G 4 For
ATCCGTCACACCTGCTCTAATTAGGATACGGGGCAACAGAACGAGAGG
GGGGAATGGTGTTGGCTCCCGTAT
G 5 For
ATCCGTCACACCTGCTCTTGAGTCAAAGAGTTTAGGGAGGAGCTAACA
TAACAGTGGTGTTGGCTCCCGTAT
G 7 For
ATCCGTCACACCTGCTCTAACAACAATGCATCAGCGGGCTGGGAACGC
ATGCGGTGGTGTTGGCTCCCGTAT
G 8 For
ATCCGTCACACCTGCTCTGAACAGGTTATAAGCAGGAGTGATAGTTTC
AGGATCTGGTGTTGGCTCCCGTAT
G 9 For
ATCCGTCACACCTGCTCTCGGCGGCTCGCAAACCGAGTGGTCAGCACC
CGGGTTGGTGTTGGCTCCCGTAT
G 10 For
ATCCGTCACACCTGCTCTGCGCAAGACGTAATCCACAAGACCGTGAAA
ACATAGTGGTGTTGGCTCCCGTAT

Glucosamine (from LPS) Reverse Sequences:

G 1 Rev
ATACGGGAGCCAACACCATTCCCCCCTCTCGTTCTGTTGCCCCGTATCCT
AATTAGAGCAGGTGTGACGGAT
G 2 Rev
ATACGGGAGCCAACACCAGCCGGATATCCGATGTGCTTGTCTGACCTGGT
CCGAGAGCAGGTGTGACGGAT
G 4 Rev
ATACGGGAGCCAACACCATTCCCCCCTCTCGTTCTGTTGCCCCGTATCCT
AATTAGAGCAGGTGTGACGGAT
G 5 Rev
ATACGGGAGCCAACACCACTGTTATGTTAGCTCCTCCCTAAACTCTTTGA
CTCAAGAGCAGGTGTGACGGAT
G 7 Rev
ATACGGGAGCCAACACCACCGCATGCGTTCCCAGCCCGCTGATGCATTGT
TGTTAGAGCAGGTGTGACGGAT
G 8 Rev
ATACGGGAGCCAACACCAGATCCTGAAACTATCACTCCTGCTTATAACCT
GTTCAGAGCAGGTGTGACGGAT
G 9 Rev
ATACGGGAGCCAACACCAACCCGGGTGCTGACCACTCGGTTTGCGAGCCG
CCGAGAGCAGGTGTGACGGAT
G 10 Rev
ATACGGGAGCCAACACCACTATGTTTTCACGGTCTTGTGGATTACGTCTT
GCGCAGAGCAGGTGTGACGGAT

KDO Antigen from LPS (Forward Primed):

K 2 For
ATCCGTCACACCTGCTCTAGGCGTAGTGACTAAGTCGCGCGAAAATCACA
GCATTGGTGTTGGCTCCCGTAT
K 5 For
ATCCGTCACACCTGCTCTCAGCGGCAGCTATACAGTGAGAACGGACTAGT
GCGTTGGTGTTGGCTCCCGTAT
K 7 For
ATCCGTCACACCTGCTCTGGCAAATAATACTAGCGATGATGGATCTGGAT
AGACTGGTGTTGGCTCCCGTAT
K 8 For
ATCCGTCACACCTGCTCTGGGGGTGCGACTTAGGGTAAGTGGGAAAGACG
ATGCTGGTGTTGGCTCCCGTAT
K 9 For
ATCCGTCACACCTGCTCTCAAGAGGAGATGAACCAATCTTAGTCCGACAG
GCGGTGGTGTTGGCTCCCGTAT
K 10 For
ATCCGTCACACCTGCTCTGGCCCGGAATTGTCATGACGTCACCTACACCT
CCTGTGGTGTTGGCTCCCGTAT

KDO Antigen from LPS (Reverse Primed):

K 2 Rev
ATACGGGAGCCAACACCAATGCTGTGATTTTCGCGCGACTTAGTCACTAC
GCCTAGAGCAGGTGTGACGGAT
K 5 Rev
ATACGGGAGCCAACACCAACGCACTAGTCCGTTCTCACTGTATAGCTGCC
GCTGAGAGCAGGTGTGACGGAT
K 7 Rev
ATACGGGAGCCAACACCAGTCTATCCAGATCCATCATCGCTAGTATTATT
TGCCAGAGCAGGTGTGACGGAT
K 8 Rev
ATACGGGAGCCAACACCAGCATCGTCTTTCCCACTTACCCTAAGTCGCAC
CCCCAGAGCAGGTGTGACGGAT
K 9 Rev
ATACGGGAGCCAACACCACCGCCTGTCGGACTAAGATTGGTTCATCTCCT
CTTGAGAGCAGGTGTGACGGAT
K 10 Rev
ATACGGGAGCCAACACCACAGGAGGTGTAGGTGACGTCATGACAATTCCG
GGCCAGAGCAGGTGTGACGGAT

### Leishmania Donovani Binding Aptamer Sequences:

Leishmania donovani Clone:940-3
Forward:
GATACGGGAGCCAACACCACCCGTATCGTTCCCAATGCACTCAGAGCAGG
TGTGACGGATG
Reverse:
CATCCGTCACACCTGCTCTGAGTGCATTGGGAACGATACGGGTGGTGTTG
GCTCCCGTATG
Leishmania donovani Clone:940-5
Forward:
GATACGGGAGCCAACACCACGTTCCCATACAAGTTACTGACAGAGCAGGT
GTGACGGATG
Reverse:
CATCCGTCACACCTGCTCTGTCAGTAACTTGTATGGGAACGTGGTGTTGG
CTCCCGTATC

Whole LPS from E. coli O111:B4 Binding Aptamer Sequences (Forward Primed):

LPS 1 For
ATCCGTCACCCCTGCTCTCGTCGCTATGAAGTAACAAAGATAGGAGCAAT
CGGGTGGTGTTGGCTCCCGTAT
LPS 3 For
ATCCGTCACACCTGCTCTAACGAAGACTGAAACCAAAGCAGTGACAGTGC
TGAATGGTGTTGGCTCCCGTAT
LPS 4 For
ATCCGTCACACCTGCTCTCGGTGACAATAGCTCGATCAGCCCAAAGTCGT
CAGATGGTGTTGGCTCCCGTAT
LPS 6 For
ATCCGTCACACCTGCTCTAACGAAATAGACCACAAATCGATACTTTATGT
TATTGGTGTTGGCTCCCGTAT
LPS 7 For
ATCCGTCACACCTGCTCTGTCGAATGCTCTGCCTGGAAGAGTTGTTAGCA
GGGATGGTGTTGGCTCCCGTAT
LPS 8 For
ATCCGTCACACCTGCTCTTAAGCCGAGGGGTAAATCTAGGACAGGGGTCC
ATGATGGTGTTGGCTCCCGTAT
LPS 9 For
ATCCGTCACACCTGCTCTACTGGCCGGCTCAGCATGACTAAGAAGGAAGT
TATGTGGTGTTGGCTCCCGTAT
LPS 10 For
ATCCGTCACACCTGCTCTGGTACGAATCACAGGGGATGCTGGAAGCTTGG
CTCTTGGTGTTGGCTCCCGTAT

Whole LPS from E. coli O111:B4 Binding Aptamer Sequences (Reverse Primed):

LPS 1 Rev
ATACGGGAGCCAACACCACCCGATTGCTCCTATCTTTGTTACTTCATAGC
GACGAGAGCAGGGGTGACGGAT
LPS 3 Rev
ATACGGGAGCCAACACCATTCAGCACTGTCACTGCTTTGGTTTCAGTCTT
CGTTAGAGCAGGTGTGACGGAT
LPS 4 Rev
ATACGGGAGCCAACACCATCTGACGACTTTGGGCTGATCGAGCTATTGTC
ACCGAGAGCAGGTGTGACGGAT
LPS 6 Rev
ATACGGGAGCCAACACCAATAACATAAAGTATCGATTTGTGGTCTATTTC
GTTAGAGCAGGTGTGACGGAT
LPS 7 Rev
ATACGGGAGCCAACACCATCCCTGCTAACAACTCTTCCAGGCAGAGCATT
CGACAGAGCAGGTGTGACGGAT
LPS 8 Rev
ATACGGGAGCCAACACCATCATGGACCCCTGTCCTAGATTTACCCCTCGG
CTTAAGAGCAGGTGTGACGGAT
LPS 9 Rev
ATACGGGAGCCAACACCACATAACTTCCTTCTTAGTCATGCTGAGCCGGC
CAGTAGAGCAGGTGTGACGGAT
LPS 10 Rev
ATACGGGAGCCAACACCAAGAGCCAAGCTTCCAGCATCCCCTGTGATTCG
TACCAGAGCAGGTGTGACGGAT

### Methylphosphonic Acid (MPA) Binding Aptamer Sequences:

MPA Forward
ATACGGGAGCCAACACCATTAAATCAATTGTGCCGTGTTCCTCTTGTCTC
ATCGAGAGCAGGTTGTACGGAT
MPA Reverse
ATCCGTACAACCTGCTCTCGATGAGACAAGAGGAACACGGCACAATTGAT
TTAATGGTGTTGGCTCCCGTAT

### Malathion Binding Aptamer Sequences:

M17 Forward
ATACGGGAGCCAACACCAGCAGTCAAGAAGTTAAGAGAAAAACAATTGTG
TATAAGAGCAGGTGTGACGGAT
M17 Reverse
ATCCGTCACACCTGCTCTTATACACAATTGTTTTTCTCTTAACTTCTTGA
CTGCTGGTGTTGGCTCCCGTAT
M21 Forward
ATCCGTCACACCTGCTCTGCGCCACAAGATTGCGGAAAGACACCCGGGGG
GCTTGGTGTTGGCTCCCGTAT
M21 Reverse
ATACGGGAGCCAACACCAAGCCCCCCGGGTGTCTTTCCGCAATCTTGTGG
CGCAGAGCAGGTGTGACGGAT
M25 Forward
ATCCGTCACACCTGCTCTGGCCTTATGTAAAGCGTTGGGTGGTGTTGGCT
CCCGTAT
M25 Reverse
ATACGGGAGCCAACACCACCCAACGCTTTACATAAGGCCAGAGCAGGTGT
GACGGAT

### Poly-D-Glutamic Acid Binding Aptamer Sequences:

PDGA 2F
CATCCGTCACACCTGCTCTGGTTCGCCCCGGTCAAGGAGAGTGGTGTTGG
CTCCCGTATC
PDGA 2R
GATACGGGAGCCAACACCACTCTCCTTGACCGGGGCGAACCAGAGCAGGT
GTGACGGATG
PDGA 5F
CATCCGTCACACCTGCTCTGGATAAGATCAGCAACAAGTTAGTGGTGTTG
GCTCCCGTATC
PDGA 5R
GATACGGGAGCCAACACCACTAACTTGTTGCTGATCTTATCAGAGCAGGT
GTGACGGATG

### Rough Ra Mutant LPS Core Antigen Binding Aptamer Sequences (Forward Primed):

R 1F
ATCCGTCACACCTGCTCTCCGCACGTAGGACCACTTTGGTACACGCTCCC
GTAGTGGTGTTGGCTCCCGTAT
R 5F
ATCCGTCACACCTGCTCTACGGATGAACGAAGATTTTAAAGTCAAGCTAA
TGCATGGTGTTGGCTCCCGTAT
R 6F
ATCCGTCACACCTGCTCTGTAGTGAAGAGTCCGCAGTCCACGCTGTTCAA
CTCATGGTGTTGGCTCCCGTAT
R 7F
ATCCGTCACACCTGCTCTACCGGCTGGCACGGTTATGTGTGACGGGCGAA
GATATGGTGTTGGCTCCCGTAT
R 8F
ATCCGTCACACCTGCTCTACCGGCTGGCACGGTTATGTGTGACGGGCGAA
GATATGGTGTTGGCTCCCGTAT
R 9F
ATCCGTCACACCTGCTCTGCGTGTGGAGCGCCTAGGTGAGTGGTGTTGGC
TCCCGTAT
R 10F
ATCCGTCACACCTGCTCTGATGTCCCTTTGAAGAGTTCCATGACGCTGGC
TCCTTGGTGTTGGCTCCCGTAT

### Rough Ra Mutant LPS Core Antigen Binding Aptamer Sequences (Reverse Primed):

R 1R
ATACGGGAGCCAACACCACTACGGGAGCGTGTACCAAAGTGGTCCTACGT
GCGGAGAGCAGGTGTGACGGAT
R 5R
ATACGGGAGCCAACACCATGCATTAGCTTGACTTTAAAATCTTCGTTCAT
CCGTAGAGCAGGTGTGACGGAT
R 6R
ATACGGGAGCCAACACCATGAGTTGAACAGCGTGGACTGCGGACTCTTCA
CTACAGAGCAGGTGTGACGGAT
R 7R
ATACGGGAGCCAACACCATATCTTCGCCCGTCACACATAACCGTGCCAGC
CGGTAGAGCAGGTGTGACGGAT
R 8R
ATACGGGAGCCAACACCATATCTTCGCCCGTCACACATAACCGTGCCAGC
CGGTAGAGCAGGTGTGACGGAT
R 9R
ATACGGGAGCCAACACCACTCACCTAGGCGCTCCACACGCAGAGCAGGTG
TGACGGAT
R 10R
ATACGGGAGCCAACACCAAGGAGCCAGCGTCATGGAACTCTTCAAAGGGA
CATCAGAGCAGGTGTGACGGAT

### Soman Binding Aptamer Sequences:

Soman 20F
ATACGGGAGCCAACACCATAGTGTTGGGCCAATACGGTAACGTGTCCTTG
GAGAGCAGGTGTGACGGAT
Soman 20R
ATCCGTCACACCTGCTCTCCAAGGACACGTTACCGACGAATTGGCCCAAC
ACTATGGTGTTGGCTCCCGTAT
Soman 23F
ATACGGGAGCCAACACCACACATACGAGTTATCTCGAGTAGAGCATGTTT
TGCCAGAGCAGGTGTGACGGAT
Soman 23R
ATCCGTCACACCTGCTCTGGCAAAACATGCTCTACTCGAGATAACTCGTA
TGTGTGGTGTTGGCTCCCGTAT
Soman 24F
ATACGGGAGCCAACACCAGGCCATCTATTGTTCGTTTTTCTATTTATCTC
ACCCAGAGCAGGTGTGACGGAT
Somna 24R
ATCCGTCACACCTGCTCTGGGTGAGATAAATAGAAAAACGAACAATAGAT
GGCCTGGTGTTGGCTCCCGTAT
Soman 25F
ATACGGGAGCCAACACCACACATACGAGTTATCTCGAGTAGAGCATGTTT
TGCCAGAGCAGGTGTGACGGAT
Soman 25R
ATCCGTCACACCTGCTCTGGCAAAACATGCTCTACTCGAGATAACTCGTA
TGTGTGGTGTTGGCTCCCGTAT
Soman 28F
ATACGGGAGCCAACACCATCCATAGCTCATCTATACCCTCTTCCGAGTCC
CACCAGAGCAGGTGTGACGGAT
Soman 28R
ATCCGTCACACCTGCTCTGGTGGGACTCGGAAGAGGGTATAGATGAGCTA
TGGATGGTGTTGGCTCCCGTAT
Soman 33F
ATACGGGAGCCAACACCAGAGCAGGTGTGACGGATAGTGACGGATGCAGA
GCAGGTGTGACGGAT
Soman 33R
ATCCGTCACACCTGCTCTGCATCCGTCACTATCCGTCACACCTGCTCTGG
TGTTGGCTCCCGTAT
Soman 41F
ATACGGGAGCCAACACCACCTTATGACGCCTCAGTACCACATCGTTTAGT
CTGTAGAGCAGGTGTGACGGAT
Soman 41R
ATCCGTCACACCTGCTCTACAGACTAAACGATGTGGTACTGAGGCGTCAT
AAGGTGGTGTTGGCTCCCGTAT
Soman 45F
ATACGGGAGCCAACACCACCCGTTTTTGATCTAATGAGGATACAATATTC
GTCTAGAGCAGGTGTGACGGAT
Soman 45R
ATCCGTCACACCTGCTCTAGACGAATATTGTATCCTCATTAGATCAAAAA
CGGGTGGTGTTGGCTCCCGTAT
Soman 46F
ATACGGGAGCCAACACCATCGAGCTCCTTGGCCCCGTTAGGATTAACGTG
ATGTAGAGCAGGTGTGACGGAT
Soman 46R
ATCCGTCACACCTGCTCTACATCACGTTAATCCTAACGGGGCCAAGGAGC
TCGATGGTGTTGGCTCCCGTAT
Soman 47F
ATACGGGAGCCAACACCATCAGAACCAAATATACATCTTCCTATGATATG
GTGGAGAGCAGGTGTGACGGAT
Soman 47R
ATCCGTCACACCTGCTCTCCACCATATCATAGGAAGATGTATATTTGGTT
CTGATGGTGTTGGCTCCCGTAT
Soman 48F
ATACGGGAGCCAACACCACACGATTGCTCCTCTCATTGTTACTTCATAGC
GACGAGAGCAGGTGTGACGGAT
Soman 48R
ATCCGTCACACCTGCTCTCGTCGCTATGAAGTAACAATGAGAGGAGCAAT
CGTGTGGTGTTGGCTCCCGTAT

### Teichoic Acid or Lipoteichoic Acid Binding Aptamer Sequences:

T5 F
GATACGGGACGACACCACACTATGGGTCGTTTAGCATCAAGGCTAGCCAA
GCCAGCAGAGGTGTGGTGAATG
T5 R
CATTCACCACACCTCTGCTGGCTTGGCTAGCCTTGATGCTAAACGACCCA
TAGTGTGGTGTCGTCCCGTATC
T6 F
CATTCACCACACCTCTGCTGGAGGAGGAAGTGGTCTGGAGTTACTTGACA
TAGTGTGGTGTCGTCCCGTATC
T6 R
GATACGGGACGACACCACACTATGTCAAGTAACTCCAGACCACTTCCTCC
TCCAGCAGAGGTGTGGTGAATG
T7 F
CATTCACCACACCTCTGCTGGACGGAAACAATCCCCGGGTACGAGAATCA
GGGTGTGGTGTCGTCCCGTATC
T7 R
GATACGGGACGACACCACACCCTGATTCTCGTACCCGGGGATTGTTTCCG
TCCAGCAGAGGTGTGGTGAATG
T9 F
CATTCACCACACCTCTGCTGGAAACCTACCATTAATGAGACATGATGCGG
TGGTGTGGTGTCGTCCCGTATC
T9 R
GATACGGGACGACACCACACCACCGCATCATGTCTCATTAATGGTAGGTT
TCCAGCAGAGGTGTGGTGAATG
E. coli O157 lipopolysaccharide (LPS)
E-5F
ATCCGTCACACCTGCTCTGGTGGAATGGACTAAGCTAGCTAGCGTTTTAA
AAGGTGGTGTTGGCTCCCGTAT
E-11F
ATCCGTCACACCTGCTCTGTAAGGGGGGGGAATCGCTTTCGTCTTAAGAT
GACATGGTGTTGGCTCCCGTAT
E-12F
ATCCGTCACACCTGCTCTGCCGGACCATCCAATATCAGCTGTGGTGTTGG
CTCCCGTAT(59)
E-16F
ATCCGTCACACCTGCTCTATCCGTCACGCCTGCTCTATCCGTCACACCTG
CTCTGGTGTTGGCTCCCGTAT
E-17F
ATCCGTCACACCTGCTCTATCAAATGTGCAGATATCAAGACGATTTGTAC
AAGATGGTGTTGGCTCCCGTAT
E-18F
ATCCGTCACACCTGCTCTGTAGATGGCAAGGCATAAGCGTCCGGAACGAT
AGAATGGTGTTGGCTCCCGTAT
E-19F
ATCCGTCACACCTGCTCTGTAGATGGCAAGGCATAAGCGTCCGGAACGAT
AGAATGGTGTTGGCTCCCGTAT
E-5R
ATACGGGAGCCAACACCACCTTTTAAAACGCTAGCTAGCTTAGTCCATTC
CACCAGAGCAGGTGTGACGGAT
E-11R
ATACGGGAGCCAACACCATGTCATCTTAAGACGAAAGCGATTCCCCCCCC
TTACAGAGCAGGTGTGACGGAT
E-12R
ATACGGGAGCCAACACCACAGCTGATATTGGATGGTCCGGCAGAGCAGGT
GTGACGGAT
E-16R
ATACGGGAGCCAACACCAGAGCAGGTGTGACGGATAGAGCAGGCGTGACG
GATAGAGCAGGTGTGACGGAT
E-17R
ATACGGGAGCCAACACCATCTTGTACAAATCGTCTTGATATCTGCACATT
TGATAGAGCAGGTGTGACGGAT
E-18R
ATACGGGAGCCAACACCATTCTATCGTTCCGGACGCTTATGCCTTGCCAT
CTACAGAGCAGGTGTGACGGAT
E-19R
ATACGGGAGCCAACACCATTCTATCGTTCCGGACGCTTATGCCTTGCCAT
CTACAGAGCAGGTGTGACGGAT
Listeriolysin (A surface protein on Listeria
monocytogenes)
LO-10F
ATCCGTCACACCTGCTCTGCCGGACCATCCAATATCAGCTGTGGTGTTGG
CTCCCGTAT
LO-11F
ATCCGTCACACCTGCTCTGGTGGAATGGACTAAGCTAGCTAGCGTTTTAA
AAGGTGGTGTTGGCTCCCGTAT
LO-13F
ATCCGTCACACCTGCTCTTAAAGTAGAGGCTGTTCTCCAGACGTCGCAGG
AGGATGGTGTTGGCTCCCGTAT
LO-15F
ATCCGTCACACCTGCTCTGTAGATGGCAAGGCATAAGCGTCCGGAACGAT
AGAATGGTGTTGGCTCCCGTAT
LO-16F
ATCCGTCACACCTGCTCTGTAGATGGCAAGGCATAAGCGTCCGGAACGAT
AGAATGGTGTTGGCTCCCGTAT
LO-17F
ATACGGGAGCCAACACCA
CAGCTGATATTGGATGGTCCGGCAGAGCAGGTGTGACGGAT
LO-19F
ATCCGTCACACCTGCTCTTGGGCAGGAGCGAGAGACTCTAATGGTAAGCA
AGAATGGTGTTGGCTCCCGTAT
LO-20F
ATCCGTCACACCTGCTCTCCAACAAGGCGACCGACCGCATGCAGATAGCC
AGGTTGGTGTTGGCTCCCGTAT
LO-10R
ATACGGGAGCCAACACCACAGCTGATATTGGATGGTCCGGCAGAGCAGGT
GTGACGGAT
LO-11R
ATACGGGAGCCAACACCACCTTTTAAAACGCTAGCTAGCTTAGTCCATTC
CACCAGAGCAGGTGTGACGGAT
LO-13R
ATACGGGAGCCAACACCATCCTCCTGCGACGTCTGGAGAACAGCCTCTAC
TTTAAGAGCAGGTGTGACGGAT
LO-15R
ATACGGGAGCCAACACCATTCTATCGTTCCGGACGCTTATGCCTTGCCAT
CTACAGAGCAGGTGTGACGGAT
LO-16R
ATACGGGAGCCAACACCATTCTATCGTTCCGGACGCTTATGCCTTGCCAT
CTACAGAGCAGGTGTGACGGAT
LO-17R
ATCCGTCACACCTGCTCTGCCGGACCATCCAATATCAGCTGTGGTGTTGG
CTCCCGTAT
LO-19R
ATACGGGAGCCAACACCATTCTTGCTTACCATTAGAGTCTCTCGCTCCTG
CCCAAGAGCAGGTGTGACGGAT
LO-20R
ATACGGGAGCCAACACCAACCTGGCTATCTGCATGCGGTCGGTCGCCTTG
TTGGAGAGCAGGTGTGACGGAT
Listeriolysin (Alternate form of Listeria surface
protein designated “Pest-Free”)
LP-3F
ATCCGTCACACCTGCTCTGTAGATGGCAAGGCATAAGCGTCCGGAACGAT
AGAATGGTGTTGGCTCCCGTAT
LP-11F
ATCCGTCACACCTGCTCTAACCAAAAGGGTAGGAGACCAAGCTAGCGATT
TGGATGGTGTTGGCTCCCGTAT
LP-13F
ATCCGTCACACCTGCTCTGCCGGACCATCCAATATCAGCT
GTGGTGTTGGCTCCCGTAT
LP-14F
ATCCGTCACACCTGCTCTGAAGCCTAACGGAGAAGATGGCCCTACTGCCG
TAGGTGGTGTTGGCTCCCGTAT
LP-15F
ATCCGTCACACCTGCTCTACTAAACAAGGGCAAACTGTAAACACAGTAGG
GGCGTGGTGTTGGCTCCCGTAT
LP-17F
ATCCGTCACACCTGCTCTGGTGTTGGCTCCCGTATAGCTTGGCTCCCGTA
TGGTGTTGGCTCCCGTAT
LP-18F
ATCCGTCACACCTGCTCTGTCGCGATGATGAGCAGCAGCGCAGGAGGGAG
GGGGTGGTGTTGGCTCCCGTAT
LP-20F
ATCCGTCACACCTGCTCTGATCAGGGAAGACGCCAACACTGGTGTTGGCT
CCCGTAT
LP-3R
ATACGGGAGCCAACACCATTCTATCGTTCCGGACGCTTATGCCTTGCCAT
CTACAGAGCAGGTGTGACGGAT
LP-11R
ATACGGGAGCCAACACCATCCAAATCGCTAGCTTGGTCTCCTACCCTTTT
GGTTAGAGCAGGTGTGACGGAT
LP-13R
ATACGGGAGCCAACACCACAGCTGATATTGGATGGTCCGGCAGAGCAGGT
GTGACGGAT
LP-14R
ATACGGGAGCCAACACCACCTACGGCAGTAGGGCCATCTTCTCCGTTAGG
CTTCAGAGCAGGTGTGACGGAT
LP-15R
ATACGGGAGCCAACACCACGCCCCTACTGTGTTTACAGTTTGCCCTTGTT
TAGTAGAGCAGGTGTGACGGAT
LP-17R
ATACGGGAGCCAACACCATACGGGAGCCAAGCTATACGGGAGCCAACACC
AGAGCAGGTGTGACGGAT
LP-18R
ATACGGGAGCCAACACCACCCCCTCCCTCCTGCGCTGCTGCTCATCATCG
CGACAGAGCAGGTGTGACGGAT
LP-20R
ATACGGGAGCCAACACCAGTGTTGGCGTCTTCCCTGATCAGAGCAGGTGT
GACGGAT
Salmonella typhimurium lipopolysaccharide (LPS)
St-7F
ATCCGTCACACCTGCTCTGTCCAAAGGCTACGCGTTAACGTGGTGTTGGC
TCCCGTAT
St-10F
ATCCGTCACACCTGCTCTGGAGCAATATGGTGGAGAAACGTGGTGTTGGC
TCCCGTAT
St-11F
ATCCGTCACACCTGCTCTGCCGGACCATCCAATATCAGCTGTGGTGTTGG
CTCCCGTAT
St-15F
ATCCGTCACACCTGCTCTGAACAGGATAGGGATTAGCGAGTCAACTAAGC
AGCATGGTGTTGGCTCCCGTAT
St-16F
ATCCGTCACACCTGCTCTGGCGGACAGGAAATAAGAATGAACGCAAAATT
TATCTGGTGTTGGCTCCCGTAT
St-18F
ATCCGTCACACCTGCTCTACGCAACGCGACAGGAACATTCATTATAGAAT
GTGTTGGTGTTGGCTCCCGTAT
St-19F
ATCCGTCACACCTGCTCTCGGCTGCAATGCGGGAGAGTAGGGGGGAACCA
AACCTGGTGTTGGCTCCCGTAT
St-20F
ATCCGTCACACCTGCTCTATGACTGGAACACGGGTATCGATGATTAGATG
TCCTTGGTGTTGGCTCCCGTAT
St-7R
ATACGGGAGCCAACACCACGTTAACGCGTAGCCTTTGGACAGAGCAGGTG
TGACGGAT
St-10R
ATACGGGAGCCAACACCACGTTTCTCCACCATATTGCTCCAGAGCAGGTG
TGACGGAT
St-11R
ATACGGGAGCCAACACCACAGCTGATATTGGATGGTCCGGCAGAGCAGGT
GTGACGGAT
St-15R
ATACGGGAGCCAACACCATGCTGCTTAGTTGACTCGCTAATCCCTATCCT
GTTCAGAGCAGGTGTGACGGAT
St-16R
ATACGGGAGCCAACACCAGATAAATTTTGCGTTCATTCTTATTTCCTGTC
CGCCAGAGCAGGTGTGACGGAT
St-18R
ATACGGGAGCCAACACCAACACATTCTATAATGAATGTTCCTGTCGCGTT
GCGTAGAGCAGGTGTGACGGAT
St-19R
ATACGGGAGCCAACACCAGGTTTGGTTCCCCCCTACTCTCCCGCATTGCA
GCCGAGAGCAGGTGTGACGGAT
St-20R
ATACGGGAGCCAACACCAAGGACATCTAATCATCGATACCCGTGTTCCAG
TCATAGAGCAGGTGTGACGGAT

### BRIEF DESCRIPTION OF THE DRAWINGS

FIG. 1. is a schematic illustration that illustrates a comparison of possible nucleic acid FRET assay formats.

FIGS. 2A. and 2B. are line graphs mapping relative fluorescence intensity against the concentration of surface protein from L. donovani from various freeze-dried and reconstituted competitive FRET-aptamer assays.

FIGS. 3A. and 3B. are “lights on” competitive FRET-aptamer spectra and a line graph for E. coli bacteria using aptamers generated against various components of lipopolysaccharide (LPS) such as the rough core (Ra) antigen and the 2-keto-3-deoxyoctanate (KDO) antigen.

FIGS. 4A. and 4B. are “lights on” competitive FRET-aptamer spectra and a bar graph for Enterococcus faecalis bacteria using aptamers generated against lipoteichoic acid.

FIGS. 5A. and 5B. are “lights off” competitive FRET-aptamer spectra and line graphs in response to increasing amounts of a foot-and-mouth disease (FMD) aphthovirus surface peptide.

FIGS. 6A. and 6B. are “lights on” competitive FRET-aptamer spectra and FIG. 6C. is a line graph in response to increasing amounts of methylphosphonic acid (MPA; an organophosphorus (OP) nerve agent breakdown product).

FIGS. 7A and 7B. are Sephadex G25 size-exclusion column profiles of complexes of Alexa Fluor (AF) 546-dUTP-labeled competitive FRET-aptamers bound to BHQ-2-amino-MPA (quencher-labeled target). The fractions with the highest absorbance at 260 nm (DNA aptamer), 555 nm (AF 546), and 579 nm (BHQ-2) were pooled and used in the competitive assay for unlabeled MPA, because these fractions contain the FRET-aptamer-quencher-labeled target complexes.

### DETAILED DESCRIPTION OF THE PREFERRED EMBODIMENT

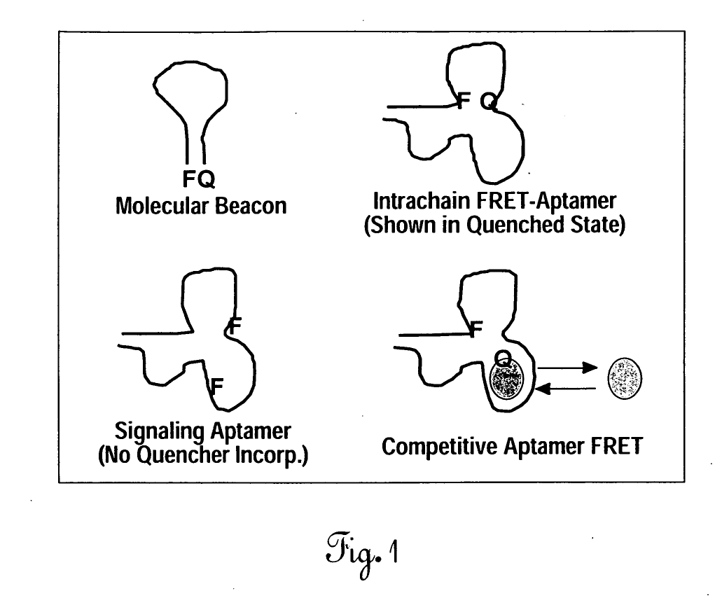

Referring to the figures, FIG. 1. provides a comparison of possible nucleic acid FRET assay formats. It illustrates how the competitive aptamer FRET scheme differs from other oligonucleotide-based FRET assay formats. Upper left is a molecular beacon (10) which may or may not be an aptamer, but is typically a short oligonucleotide used to hybridize to other DNA or RNA molecules and exhibit FRET upon hybridizing. Molecular beacons are only labeled with F and Q at the ends of the DNA molecule. Lower left is a signaling aptamer (12), which does not contain a quencher molecule, but relies upon fluorophore self-quenching or weak intrinsic quenching of the DNA or RNA to achieve limited FRET. Upper right is an intrachain FRET-aptamer (14) containing F and Q molecules built into the interior structure of the aptamer. Intrachain FRET-aptamers are naturally selected and characterized by the processes described herein. Lower right shows a competitive aptamer FRET (16) motif in which the aptamer container either F or Q and the target molecule (18) is labeled with the complementary F or Q. Introduction of unlabeled target molecules (20) then shifts the equilibrium so that some labeled target molecules are liberated from the labeled aptamer and modulate the fluorescence level of the solution up or down thereby achieving FRET. A target analyte (20) is either unlabeled or labeled with a quencher (Q). F and Q can be switched from placement in the aptamer to placement in the target analyte and vice versa.

F-labeled or Q-labeled aptamers (labeled by the polymerase chain reaction (PCR), asymmetric PCR (to produce a predominately single-stranded amplicon) using Taq, Deep Vent Exo⁻ or other heat-resistant DNA polymerases, or other enzymatic incorporation of F-NTPs or Q-NTPs) may be used in competitive or displacement type assays in which the fluorescence light levels change proportionately in response to the addition of various levels of unlabeled analyte which compete to bind with the F-labeled or Q-labeled analytes.

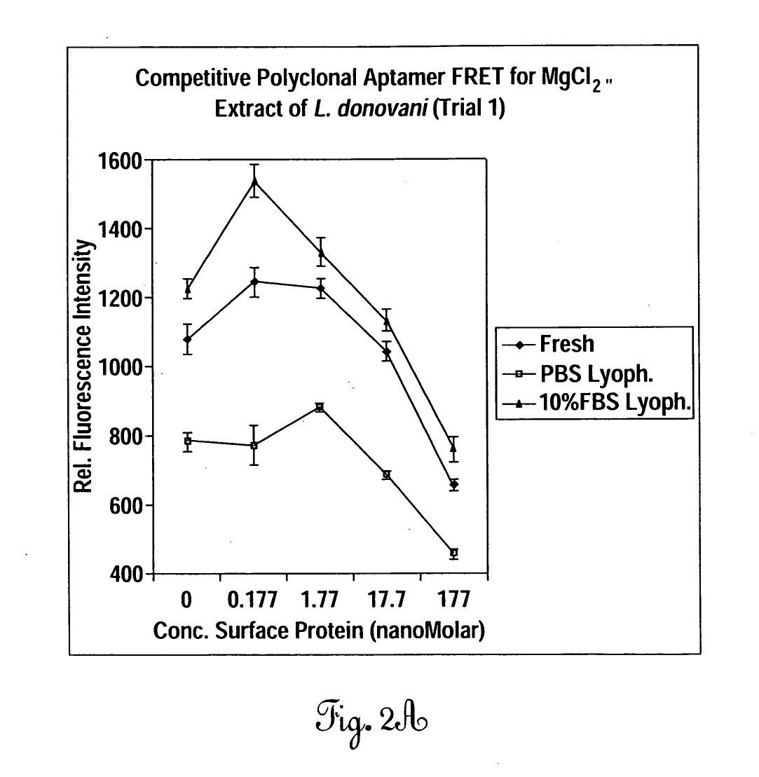

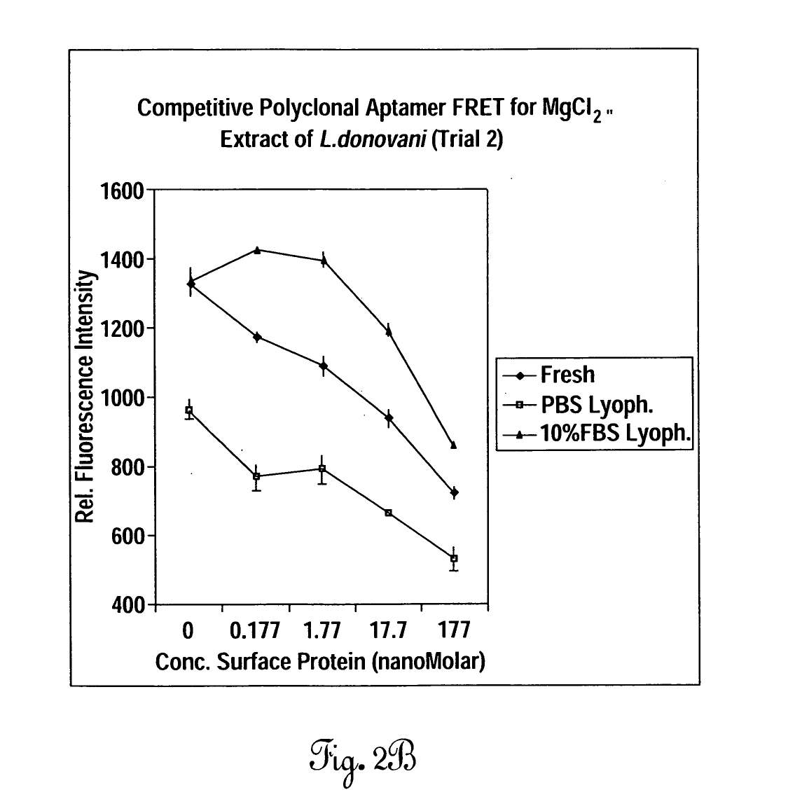

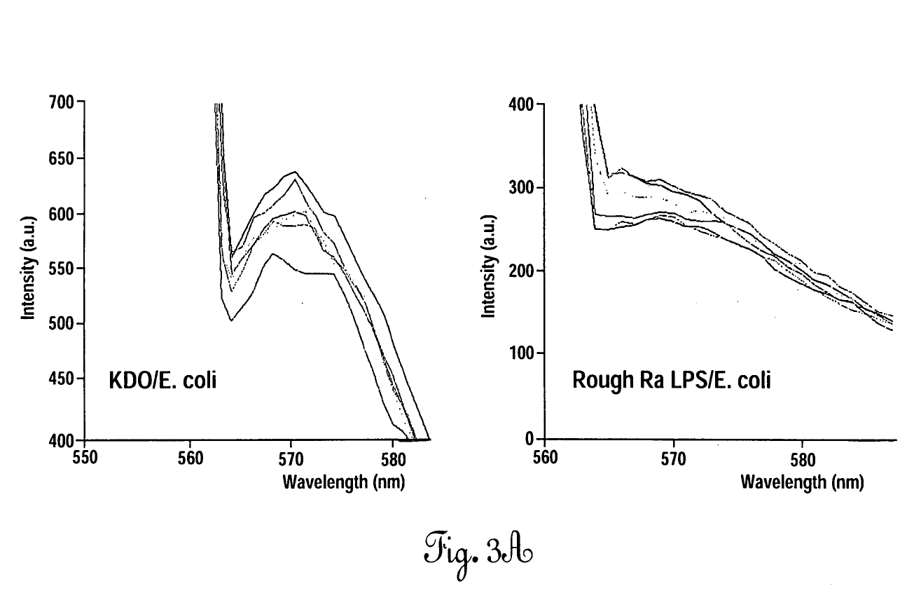

Competitive aptamer-FRET assays may be used for the detection and quantitation of small molecules (<1,000 daltons) including pesticides, acetylcholine (ACh), organophosphate (“OP”) nerve agents such as sarin, soman, and VX, OP nerve agent breakdown products such as MPA, isopropyl-MPA, ethylmethyl-MPA, pinacolyl-MPA, etc., acetylcholine (ACh), acyl homoserine lactone (AHL) and other quorum sensing (QS) molecules natural and synthetic amino acids and their derivatives (e.g., histidine, histamine, homocysteine, DOPA, melatonin, nitrotyrosine, etc.), short chain proteolysis products such as cadaverine, putrescine, the polyamines spermine and spermidine, nitrogen bases of DNA or RNA, nucleosides, nucleotides, and their cyclical isoforms (e.g., cAMP and cGMP), cellular metabolites (e.g., urea, uric acid), pharmaceuticals (therapeutic drugs), drugs of abuse (e.g., narcotics, hallucinogens, gamma-hydroxybutyrate, etc.), cellular mediators (e.g., cytokines, chemokines, immune modulators, neural modulators, inflammatory modulators such as prostaglandins, etc.), or their metabolites, explosives (e.g., trinitrotoluene) and their breakdown products or byproducts, peptides and their derivatives, macromolecules including proteins (such as bacterial surface proteins from Leishmania donovani, See FIGS. 2A and 2B), glycoproteins, lipids, glycolipids, nucleic acids, polysaccharides, lipopolysaccharides (LPS), and LPS components (e.g., ethanolamine, glucosamine, LPS-specific sugars, KDO, rough core antigens, etc.), viruses, whole cells (bacteria and eukaryotic cells, cancer cells, etc.), and subcellular organelles or cellular fractions.

If the target molecule is a larger, water-soluble molecule such as a protein, glycoprotein, or other water soluble macromolecule, then exposure of the nascent F-labeled and Q-labeled DNA or RNA random library to the free target analyte is done in solution. If the target is a soluble protein or other larger water-soluble molecule, then the optimal FRET-aptamer-target complexes are separated by size-exclusion chromatography. The FRET-aptamer-target complex population of molecules is the heaviest subset in solution and will emerge from a size-exclusion column first, followed by unbound FRET-aptamers and unbound proteins or other targets. Among the subset of analyte-bound aptamers there will be heterogeneity in the numbers of F- and Q-NTP's that are incorporated as well as nucleotide sequence differences, which will again effect the mass, electrical charge, and weak interaction capabilities (e.g., hydrophobicity and hydrophilicity) of each analyte-aptamer complex. These differences in physical properties of the aptamer-analyte complexes can then be used to separate out or partition the bound from unbound analyte-aptamer complexes.

If the target is a small molecule, then exposure of the nascent F-labeled and Q-labeled DNA or RNA random library to the target may be done by immobilizing the target. The small molecule can be immobilized on a column, membrane, plastic or glass bead, magnetic bead, quantum dot, or other matrix. If no functional group is available on the small molecule for immobilization, the target can be immobilized by the Mannich reaction (formaldehyde-based condensation reaction) on a PharmaLink™ column. Elution of bound DNA from the small molecule affinity column, membrane, beads or other matrix by use of 0.2-3.0M sodium acetate at a pH of between 3 and 7.

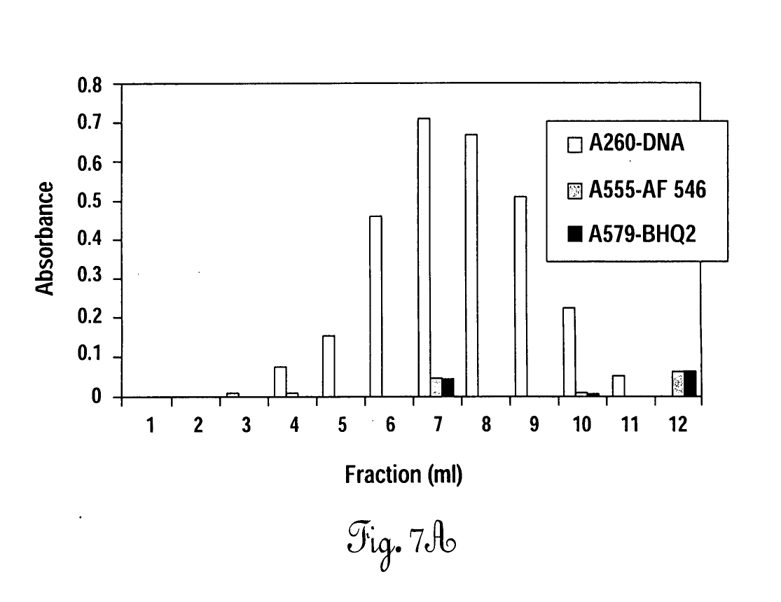

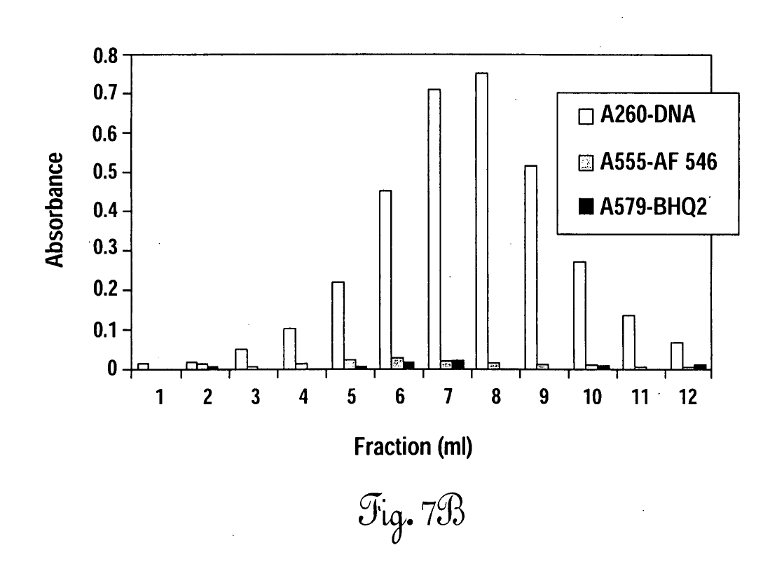

These can be separated from the non-binding doped DNA molecules by running the aptamer-protein aggregates (or selected aptamers-protein aggregates) through a size exclusion column, by means of size-exclusion chromatography using Sephadex™ or other gel materials in the column. Since they vary in weight due to variations in aptamers sequences and degree of labeling, they can be separated into fractions with different fluorescence intensities. Purification methods such as preparative gel electrophoresis are possible as well. Small volume fractions (≦1 mL) can be collected from the column and analyzed for absorbance at 260 nm and 280 nm which are characteristic wavelengths for DNA and proteins. In addition, the characteristic absorbance wavelengths for the fluorophore and quencher (FIGS. 7A and 7B) should be monitored. The heaviest materials come through a size-exclusion column first. Therefore, the DNA-protein complexes will come out of the column before either the DNA or protein alone.

Means of separating FRET-aptamer-target complexes from solution by alternate techniques (other than size-exclusion chromatography) include, without limitation, molecular weight cut off spin columns, dialysis, analytical and preparative gel electrophoresis, various types of high performance liquid chromatography (HPLC), thin layer chromatography (TLC), and differential centrifugation using density gradient materials.

The optimal (most sensitive or highest signal to noise ratio) FRET-aptamers among the bound class of FRET-aptamer-target complexes are identified by assessment of fluorescence intensity for various fractions of the FRET-aptamer-target class. The separated DNA-protein complexes will exhibit the highest absorbance at established wavelengths, such as 260 nm and 280 nm. The fractions showing the highest absorbance at the given wavelengths, such as 260 nm and 280 nm, are then further analyzed for fluorescence and those fractions exhibiting the greatest fluorescence are selected for separation and sequencing.

These similar FRET-aptamers may be further separated using techniques such as ion pair reverse-phase high performance liquid chromatography, ion-exchange chromatography (IEC, either low pressure or HPLC versions of IEC), thin layer chromatography (TLC), capillary electrophoresis, or similar techniques.

The final FRET aptamers are able to act as one-step “lights on” or “lights off” binding and detection components in assays.

Intrachain FRET-aptamers that are to be used in assays with long shelf-lives may be lyophilized (freeze-dried) and reconstituted.

FIGS. 2A. and 2B. are line graphs mapping the fluorescence intensity of the DNA aptamers against the concentration of the surface protein. The figures present results from two independent trials of a competitive aptamer-FRET assay involving fluorophore-labeled DNA aptamers and surface extracted proteins from Leishmania donovani bacteria. In this type of assay, the fluorescence intensity decreases as a function of increasing analyte concentration, and is thus referred to as a “lights off” assay. If the fluorescence intensity increases as a function of increasing analyte concentration, then it is referred to as a “lights on” assay. Also shown are translations of the assay curve up or down due to lyophilization (freeze-drying) in the absence or presence of 10% fetal bovine serum (FBS). Error bars represent the standard deviations of the mean for three measurements.

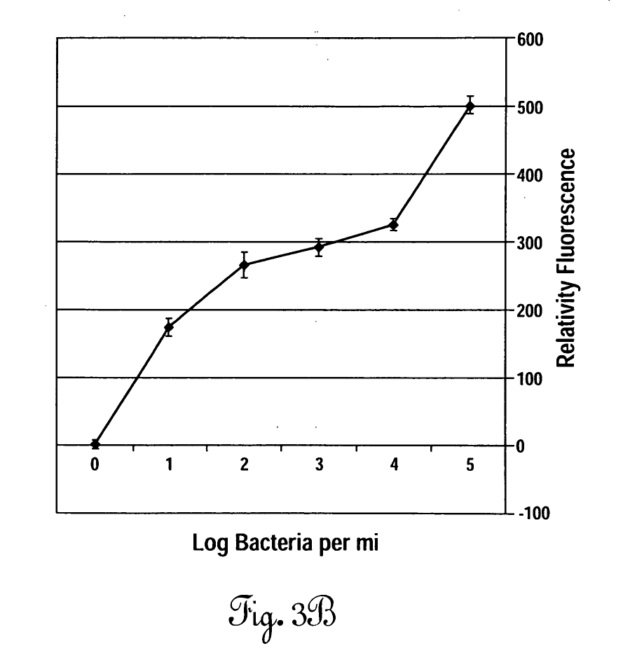

FIGS. 3A. and 3B. are FRET fluorescence spectra and line graphs generated as a function of live E. coli (Crooks strain, ATCC No. 8739) concentration using LPS component competitive FRET-aptamers. Error bars represent the standard deviations of the mean for four measurements.

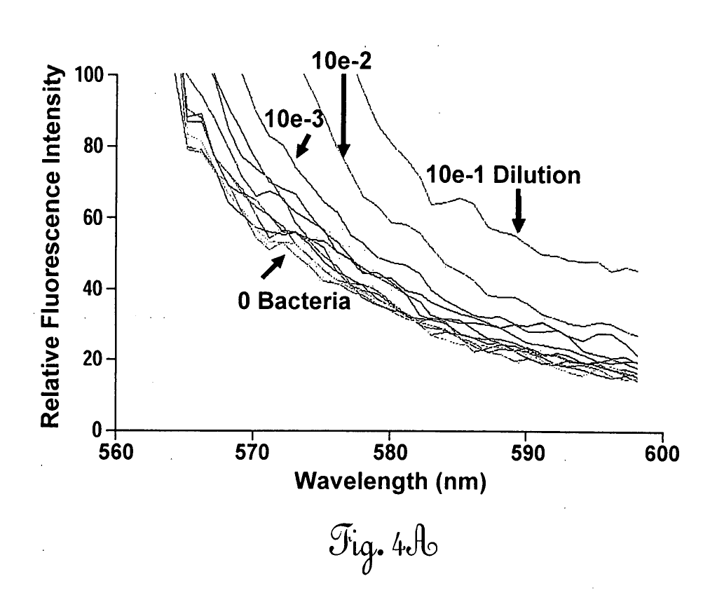

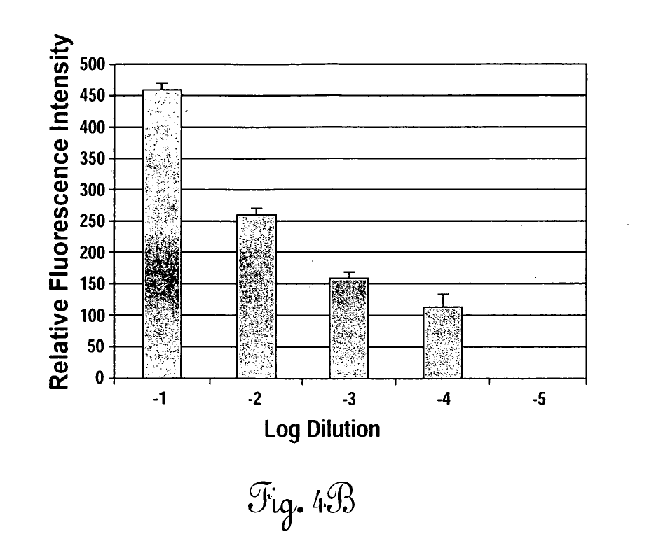

FIGS. 4A. and 4B. are FRET fluorescence spectra and line graphs generated as a function of live Enterococcus faecalis concentration using lipoteichoic acid (TA) competitive FRET-aptamers. Error bars represent the standard deviations of the mean for four measurements.

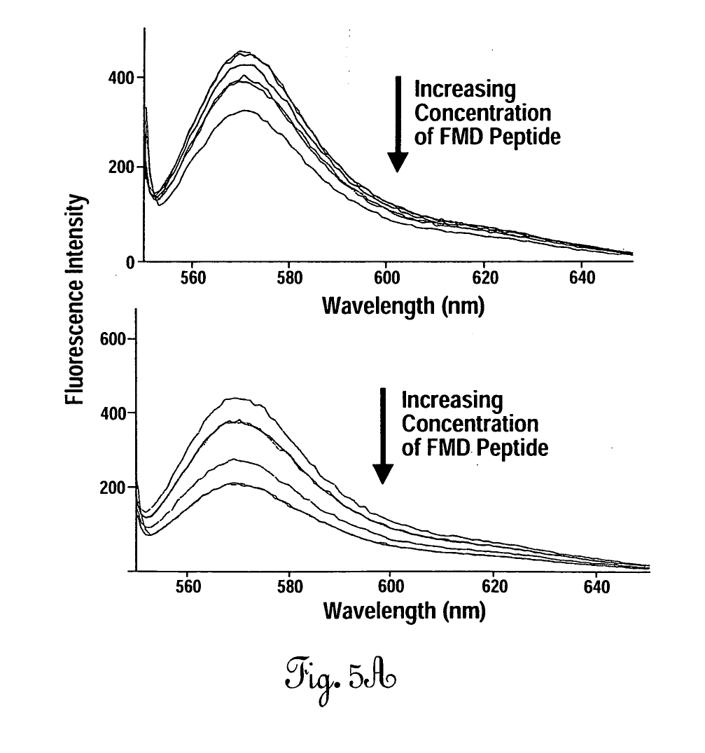

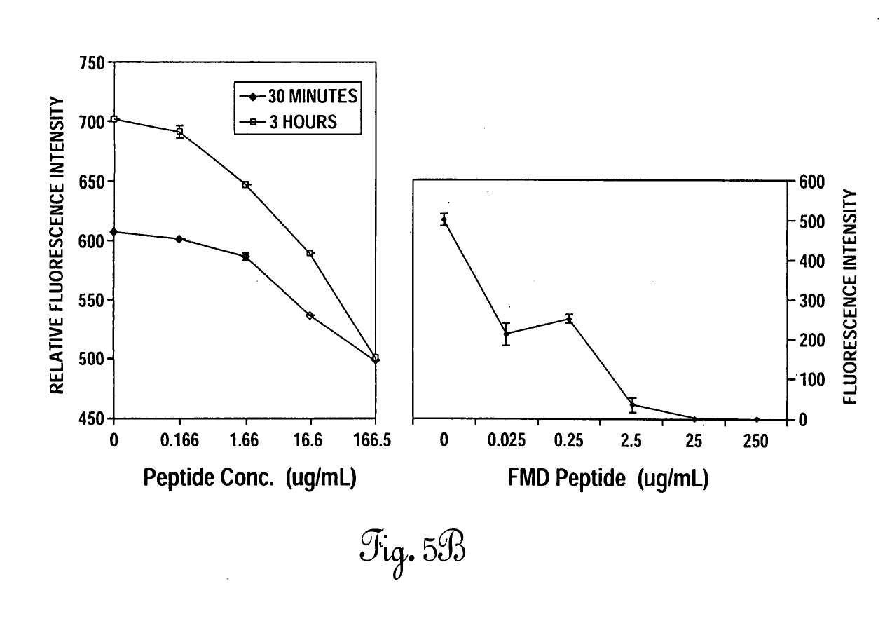

FIGS. 5A. and 5B. are FRET fluorescence spectra and line graphs generated as a function of Foot-and-Mouth Disease (FMD) peptide concentration using FMD peptide competitive FRET-aptamers. Error bars represent the standard deviations of the mean for four measurements.

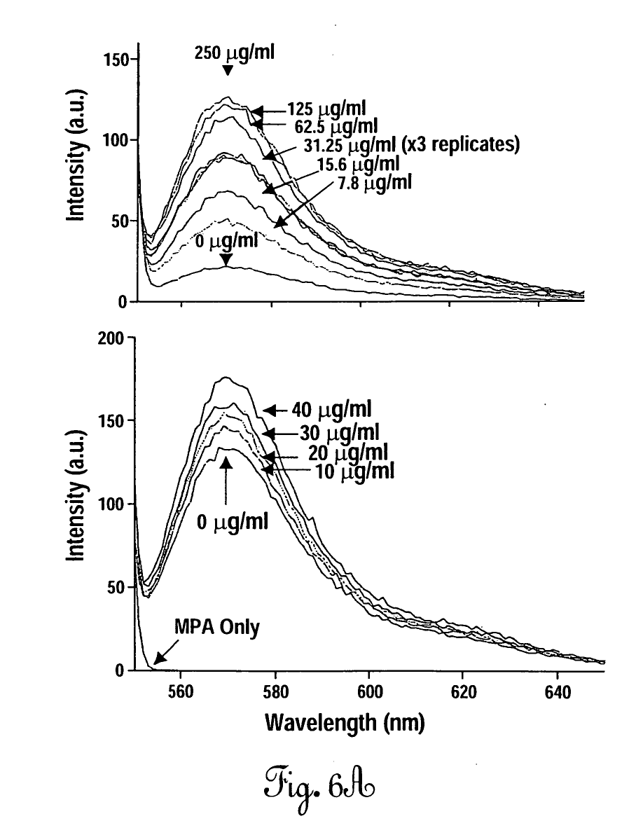

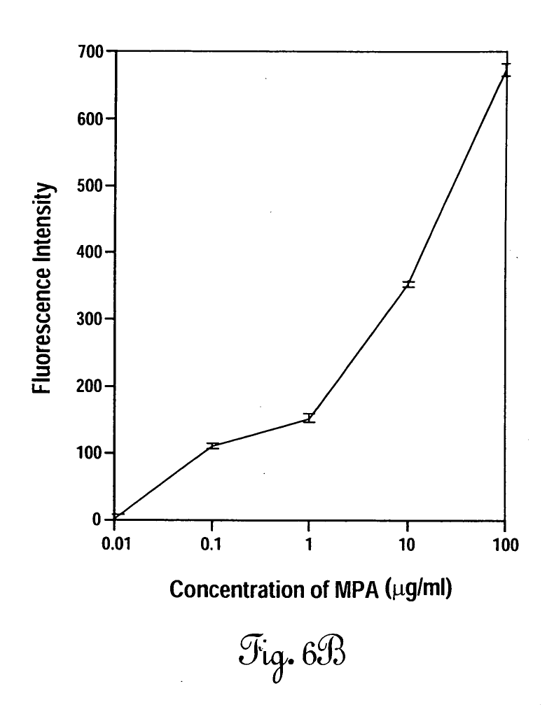

FIGS. 6A. and 6B. are FRET fluorescence spectra, and FIG. 6C. is a line graph, all generated as a function of methylphosphonic acid (MPA; OP nerve agent degradation product) concentration using MPA competitive FRET-aptamers to represent possible FRET-aptamer assays for MPA and OP nerve agents such as pesticides, sarin, soman, VX, etc. Error bars represent the standard deviations of the mean for four measurements.

FIGS. 7A. and 7B. are two independent Sephadex™ G25 elution profiles for BHQ-2-amino-MPA-AF 546-MPA aptamer complex based on absorbance peaks characteristic of the aptamer (260 nm), fluorophore (555 nm), and quencher (579 nm) to assess the optimal fraction for competitive FRET-aptamer assay of MPA shown in FIG. 6. Similar elution profiles can be expected for all such soluble targets when the target is quencher-labeled and complexed to a fluorophore-labeled aptamer.

### Example 1

### Competitive Aptamer-FRET Assay for Surface Proteins Extracted from Bacteria (L. donovani)

In this example, surface proteins from heat-killed Leishmania donovani were extracted with 3 M MgCl₂ overnight at 4° C. These proteins were then linked to tosyl-magnetic microbeads and used in a standard SELEX aptamer generation protocol. After 5 rounds of SELEX, the aptamer population was “doped” during the standard PCR reaction with 3 uM fluorescein-dUTP and purified on 10 kD molecular weight cut off spin columns. Some of the L. donovani surface proteins were then labeled with dabcyl-NHS ester and purified on a PD-10 (Sephadex G25) column. The dabcyl-labeled surface proteins were combined with the fluorescein-labeled aptamer population so as to produce a 1:1 fluorescein-aptamer:dabcyl-protein ratio. Thereafter, unlabeled L. donovani surface proteins were introduced into the assay system to compete with the labeled proteins for binding to the aptamers, thereby producing the “lights off” FRET assay results depicted in FIGS. 2A and 2B (fresh assay results, solid line). The assays were also examined following lyophilization (freeze drying) and reconstitution (rehydration) in the presence or absence of 10% fetal bovine serum (FBS) as a possible preservative with the results shown in FIGS. 2A and 2B. The DNA sequences of several of these candidate Leishmania aptamers are given in SEQ IDs XX-XX.

### Example 2

### Competitive FRET-Aptamer Assay for E. coli in Environmental Water Samples or Foods Using LPS Component Aptamers

E. coli, especially the enterohemorrhagic strains such as O157:H7 which produce Verotoxin or Shiga toxins, are of concern in environmental water samples and foods. Their rapid detection (within minutes) with ultrasensitivity is important in protecting swimmers as well as those consuming water and foods. In this example, aptamers were generated against whole LPS from E. coli O111:B4 and its components such as glucosamine, KDO, and the rough mutant core antigen (Ra; lacking the outer oligosaccharide chains). In the case of glucosamine, the free primary amine in its structure enabled conjugation to tosyl-magnetic beads. KDO antigen was immobilized onto amine-conjugated magnetic beads via its carboxyl group and the bifunctional linker EDC. The rough Ra core antigen and whole LPS were linked to amine-magnetic beads via reductive amination using sodium periodate to oxidize the saccharides to aldehydes followed by the use of sodium cyanoborohydride for reductive amination as will be clear to anyone skilled in the art. Once immobilized the target-magnetic beads were used for aptamer affinity selection from a random library of 72 base aptamers (randomized 36 mer flanked by known 18 mer primer regions). After 5 rounds of aptamer selection and amplification, the various LPS component aptamer populations were subjected to 10 rounds of PCR in the presence of Alexa Fluor (AF) 546-14-dUTP (Invitrogen), then heated to 95° C. for 5 minutes and added to heat-killed E. coli O157:H7 (Kirkegaard Perry Laboraties, Inc., Gaithersburg, Md.) and used in competitive FRET-aptamer assays with various concentrations of unlabeled live E. coli (Crooks strain, ATCC No. 8739) resulting in the FRET spectra and line graphs shown in FIGS. 3A and 3B. Candidate DNA aptamer sequences for detection of LPS O111 and LPS components or associated E. coli and other Gram negative bacteria are given in SEQ ID Nos. XX-XX.

### Example 3

### Competitive FRET-Aptamer Assay for Enterococci in Environmental Water Samples

Gram positive enterococci, such as Enterococcus faecalis, are also indicators of fecal contamination of environmental water, recreational waters, or treated wastewater (effluent from sewage treatment plants). Water testers desire to detect the presence of these bacteria rapidly (within minutes) and with great sensitivity. In this example, aptamers were generated against whole lipoteichoic acid (TA; teichoic acid). TA from E. faecalis was immobilized on magnetic beads by reductive amination using sodium periodate to first oxidize saccharides into aldehydes followed by reductive amination using amine-magnetic beads and sodium cyanoborohydride as will be known to anyone skilled in the art. Once immobilized the target-magnetic beads were used for aptamer affinity selection from a random library of 72 base aptamers (randomized 36 mer flanked by known 18 mer primer regions). After 5 rounds of aptamer selection and amplification, the TA aptamer population was subjected to 10 rounds of PCR in the presence of AF 546-14-dUTP (Invitrogen), then heated to 95° C. for 5 minutes and added to live E. faecalis. The complexes were purified by centrifugation and washing and used in competitive FRET-aptamer assays with various concentrations of unlabeled live E. faecalis resulting in the FRET spectra and bar graphs shown in FIGS. 4A. and 4B. Candidate DNA aptamer sequences for detection of lipoteichoic acid (TA) and associated enterococi or other Gram positive bacteria are given in SEQ ID Nos. XX-XX.

### Example 4

### Detection of Foot-and-Mouth (FMD) Disease or Other Highly Communicable Viruses Among Animal or Human Populations

FMD has not existed in the United States for decades, but if it were reintroduced via agricultural bioterrorism or accidental means, it could cripple the multi-billion dollar livestock industry. Hence, rapid detection of FMD in the field (on farms) is of great value in quarantining infected animals or farms and limiting the spread of FMD. Likewise, epidemiologists have many uses for rapid field detection and identification of viruses and other microbes such as influenzas, potential small pox outbreaks, etc. which FRET-aptamer assays could satisfy. A highly conserved peptide from the VP1 structural protein of O-type FMD, which is widely distributed throughout the world, was chosen as the aptamer development target. The peptide had the following primary amino acid sequence: RHKQKIVAPVKQLL. This sequence corresponds to amino acids 200 through 213 of 16 different O-type FMD viruses and represents a neutralizable antigenic region wherein antibodies are known to bind. The FMD peptide was immobilized on tosyl-magnetic beads via the three lysine residues in its structure. Once immobilized the target-magnetic beads were used for aptamer affinity selection from a random library of 72 base aptamers (randomized 36 mer flanked by known 18 mer primer regions). After 5 rounds of aptamer selection and amplification, the FMD (peptide) aptamer populations were subjected to 10 rounds of PCR in the presence of Alexa Fluor (AF) 546-14-dUTP (Invitrogen), then heated to 95° C. for 5 minutes and added to their BHQ-2-labeled-peptide target. The complexes were purified by size-exclusion chromatography over Sephadex G25 and used in competitive FRET-aptamer assays with various concentrations of unlabeled FMD peptide resulting in the FRET spectra and line graphs shown in FIGS. 5A and 5B. Candidate DNA aptamer sequences for detection of the FMD peptide and associated strains of FMD virus are given in SEQ ID Nos. XX-XX.

### Example 5

### Detection of Organophosphorus (OP) Nerve Agent, Pesticides, and Acetylcholine (ACh)

The use of OP nerve agents on Iraqi Kurds in the late 1980's followed by the 1995 use of sarin in a Japanese subway underscore the need for rapid and sensitive detection of OP nerve agents such as FRET-aptamer assays might provide. In addition, there is a desire in the agricultural industry to detect pesticides (also OP nerve agents) on the surfaces of fruits and vegetables in the field or in grocery stores. Finally, aptamers that bind and detect acetylcholine (ACh) may be of value in determining the impact of OP nerve agents on acetylcholinesterase (AChE) activity. Candidate aptamer sequences for the nerve agent soman, methylphosphonic acid (MPA, a common nerve agent hydrolysis product), the pesticides diazinon and malathion, and ACh are given in SEQ ID Nos. XX-XX. Amino-MPA and para-aminophenyl-soman were immobilized on tosyl-magnetic beads and used for aptamer selelction. ACh and the pesticides were immobilized onto PharmaLink™ (Pierce Chemical Co.) affinity columns by the Mannich formaldehyde condensation reaction and used for aptamer selection. The polyclonal or monoclonal candidate MPA aptamers were labeled with AF 546-14-dUTP by 10 rounds of conventional PCR or 20 rounds of asymmetric as appropriate with Deep Vent Exo⁻ polymerase and then complexed to BHQ-2-amino-MPA. The complexes were purified by size-exclusion chromatography over Sephadex G-15 and used to generate FRET spectra and line graphs as a function of unlabeled MPA as shown in FIGS. 6A, 6B, and 6C.

Other potential examples of uses for competitive FRET-aptamer assays include, but are not limited to:

1) Detection and quantitation of quorum sensing (QS) molecules such as acyl homoserine lactones (AHLs such as N-Decanoyl-DL-Homoserine Lactone; SEQ ID Nos. XX-XX), which communicate between many Gram negative bacteria such as Pseudomonads to signal proliferation and the induction of virulence factors, thereby leading to disease.
2) Detection and quantitation of botulinum toxins (BoNTs) for determination of the presence of biological warfare or bioterrorism agents (SEQ ID Nos. XX-XX) and Clostridium botulinum in vivo.
3) Detection and quantitation of Campylobacter jejuni and related Campylobacter species (SEQ ID Nos. XX-XX) in foods and water to prevent foodborne or waterborne illness outbreaks in a 2006 JCLA paper.
4) Detection and quantitation of poly-D-glutamic acid (PDGA; SEQ ID Nos. XX-XX) from vegetative forms of pathogenic Bacillus anthracis or other similar encapsulated bacteria in vivo or in the environment to rapidly diagnose biological warfare or bioterrorist activity and provide intervention.
5) Detection and quantitation of Bacillus thuringiensis bacterial endospores in the environment to assist in biological warfare or bioterrorism detection field trials or forensic work.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

We claim:

1. A method of using a competitive type assay to determine the presence of target molecules in a solution, comprising:
incorporating a volume of a fluorophore (“F”)-labeled aptamer into said solution that may contain unlabeled target molecules, wherein said F-labeled aptamer will bind with said target molecule, and wherein said F is located in the interior portion of said aptamer;
adding labeled target molecules to said solution, wherein said labeled target molecules are labeled with a quencher (“Q”) that is complimentary to said F of said F-labeled aptamer, and wherein said Q-labeled target molecules compete with said unlabeled target molecules to bind with said F-labeled aptamers;
wherein said F and said Q are spectrally matched such that there is a detectable change in the fluorescent signal of said aptamer when said F and said Q are moved into or out of functional proximity;
wherein fluorescence light levels change proportionately in response to the amount of said Q-labeled target molecules that are able to bind with said F-labeled aptamers;
measuring said fluorescence light level in order to determine the presence of said unlabeled target molecules in said solution;
wherein said aptamer has a binding pocket into which said target molecule binds to said aptamer; and
wherein said binding pocket is comprised of 3 to 6 nucleotides.

2. The method of claim 1 wherein said binding pocket is comprised of 3 or more nucleotides of a specific sequence or arrangement to confer the appropriate volume and conformation in 3-dimensional space to enable optimal binding to target molecules.

3. The method of claim 1 wherein said aptamer is selected from nucleotide sequences selected from the group consisting of SEQ ID NOs: 43, or a truncate thereof.

4. The method of claim 2 wherein said aptamer is selected from nucleotide sequences selected from the group consisting of SEQ ID NOs: 43, or a truncate thereof.

5. A method of using a competitive type assay to determine the presence of target molecules in a solution, comprising:
incorporating a volume of a quencher (“Q”)-labeled aptamer into a solution that may contain unlabeled target molecules, wherein said Q-labeled aptamer will bind with said target molecule, and wherein said Q is located in the interior portion of said aptamer;
adding labeled target molecules to said solution, wherein said labeled target molecules are labeled with a fluorophore (“F”) that is complimentary to said Q of said Q-labeled aptamer, and wherein said F-labeled target molecules compete with said unlabeled target molecules to bind with said Q-labeled aptamers;
wherein said F and said Q are spectrally matched such that there is a detectable change in the fluorescent signal of said aptamer when said F and said Q are moved into or out of functional proximity;
wherein fluorescence light levels change proportionately in response to the amount of said F-labeled target molecules that are able to bind with said Q-labeled aptamers;
measuring said fluorescence light level in order to determine the presence of said unlabeled target molecules in said solution;
wherein said aptamer has a binding pocket into which said target molecule binds to said aptamer; and
wherein said binding pocket is comprised of 3 to 6 nucleotides.

6. The method of claim 6 wherein said binding pocket is comprised of 3 or more nucleotides of a specific sequence or arrangement to confer the appropriate volume and conformation in 3-dimensional space to enable optimal binding to target molecules.

7. The method of claim 6 wherein said aptamer is selected from nucleotide sequences selected from the group consisting of SEQ ID NOs: 43, or a truncate thereof.

8. The method of claim 7 wherein said aptamer is selected from nucleotide sequences selected from the group consisting of SEQ ID NOs: 43, or a truncate thereof.


SEQUENCE LISTING (225 sequences)

SEQ ID NO: 1 - DNA, 72 bp, Artificial Sequence
  chemically synthesized
atacgggagccaacaccacgatacccgcttatgaattttaaattaattgtgatcagagcaggtgtgacggat

SEQ ID NO: 2 - DNA, 72 bp, Artificial Sequence
  chemically synthesized
atccgtcacacctgctctgatcacaattaatttaaaattcataagcgggtatcgtggtgttggctcccgtat

SEQ ID NO: 3 - DNA, 72 bp, Artificial Sequence
  chemically synthesized
atacgggagccaacaccaactttcacacatacttgttataccacacgatcttttagagcaggtgtgacggat

SEQ ID NO: 4 - DNA, 72 bp, Artificial Sequence
  chemically synthesized
atccgtcacacctgctctaaaagatcgtgtggtataacaagtatgtgtgaaagttggtgttggctcccgtat

SEQ ID NO: 5 - DNA, 72 bp, Artificial Sequence
  chemically synthesized
atacgggagccaacaccactttgtaactcatttgtagtttgggttgctccccctagagcaggtgtgacggat

SEQ ID NO: 6 - DNA, 72 bp, Artificial Sequence
  chemically synthesized
atccgtcacacctgctctagggggagcaacccaaactacaaatgagttacaaagtggtgttggctcccgtat

SEQ ID NO: 7 - DNA, 72 bp, Artificial Sequence
  chemically synthesized
atacgggagccaacaccatttcccgcttatcttcatccactgcttagcatatgtagagcaggtgtgacggat

SEQ ID NO: 8 - DNA, 72 bp, Artificial Sequence
  chemically synthesized
atccgtcacacctgctctacatatgctaagcagtggatgaagataagcgggaaatggtgttggctcccgtat

SEQ ID NO: 9 - DNA, 72 bp, Artificial Sequence
  chemically synthesized
atacgggagccaacaccaggcactgtatcacaccgtcaagaatgtgatcccctgagagcaggtgtgacggat

SEQ ID NO: 10 - DNA, 72 bp, Artificial Sequence
  chemically synthesized
atccgtcacacctgctctcaggggatcacattcttgacggtgtgatacagtgcctggtgttggctcccgtat

SEQ ID NO: 11 - DNA, 72 bp, Artificial Sequence
  chemically synthesized
atacgggagccaacaccatgtcatttaccttcatcatgacagtgttagtatacgagagcaggtgtgacggat

SEQ ID NO: 12 - DNA, 72 bp, Artificial Sequence
  chemically synthesized
atccgtcacacctgctctaggggatcaaagctatgcgaccatgcgagtggatactggtgttggctcccgtat

SEQ ID NO: 13 - DNA, 72 bp, Artificial Sequence
  chemically synthesized
atacgggagccaacaccagttgccgcctaccttgattattctacattacccgttagagcaggtgtgacggat

SEQ ID NO: 14 - DNA, 72 bp, Artificial Sequence
  chemically synthesized
atccgtcacacctgctctaacgggtaatgtagaataatcaaggtaggcggcaactggtgttggctcccgtat

SEQ ID NO: 15 - DNA, 72 bp, Artificial Sequence
  chemically synthesized
atacgggagccaacaccagtatacatacgaagagttgaaaccaatgcttcgttcagagcaggtgtgacggat

SEQ ID NO: 16 - DNA, 72 bp, Artificial Sequence
  chemically synthesized
atccgtcacacctgctctgaacgaagcattggtttcaactcttcgtatgtatactggtgttggctcccgtat

SEQ ID NO: 17 - DNA, 72 bp, Artificial Sequence
  chemically synthesized
atacgggagccaacaccataccccgaatggctgttttcagtaccaatatgactcagagcaggtgtgacggat

SEQ ID NO: 18 - DNA, 72 bp, Artificial Sequence
  chemically synthesized
atccgtcacacctgctctgagtcatattggtactgaaaacagccattcggggtatggtgttggctcccgtat

SEQ ID NO: 19 - DNA, 72 bp, Artificial Sequence
  chemically synthesized
atacgggagccaacaccactgtcacgatcgtcgttccttttaatccttgtgtctagagcaggtgtgacggat

SEQ ID NO: 20 - DNA, 72 bp, Artificial Sequence
  chemically synthesized
atccgtcacacctgctctagacacaaggattaaaaggaacgacgatcgtgacagtggtgttggctcccgtat

SEQ ID NO: 21 - DNA, 72 bp, Artificial Sequence
  chemically synthesized
atacgggagccaacaccactggacactgaccctcgcactagctttctgacgggtagagcaggtgtgacggat

SEQ ID NO: 22 - DNA, 72 bp, Artificial Sequence
  chemically synthesized
atccgtcacacctgctctacccggccgaagaatagtgctcggtacttagtcgcgtggtgttggctcccgtat

SEQ ID NO: 23 - DNA, 72 bp, Artificial Sequence
  chemically synthesized
atacgggagccaacaccatttggactttaaatagtggactccttctttgtctcgagagcaggtgtgacggat

SEQ ID NO: 24 - DNA, 72 bp, Artificial Sequence
  chemically synthesized
atccgtcacacctgctctcgagacaaagaaggagtccactatttaaagtccaaatggtgttggctcccgtat

SEQ ID NO: 25 - DNA, 72 bp, Artificial Sequence
  chemically synthesized
atacgggagccaacaccatcatttgcaaatatgaattccacttaaagaaattcaagagcaggtgtgacggat

SEQ ID NO: 26 - DNA, 72 bp, Artificial Sequence
  chemically synthesized
atccgtcacacctgctcttgaatttctttaagtggaattcatatttgcaaatgatggtgttggctcccgtat

SEQ ID NO: 27 - DNA, 72 bp, Artificial Sequence
  chemically synthesized
atacgggagccaacaccatcctaactggtctaatttttgctgttaccgatcccgagagcaggtgtgacggat

SEQ ID NO: 28 - DNA, 72 bp, Artificial Sequence
  chemically synthesized
atccgtcactcctgctctcgggatcggtaacagcaaaaattagaccagttaggatggtgttggctcccgtat

SEQ ID NO: 29 - DNA, 72 bp, Artificial Sequence
  chemically synthesized
atacgggagccaacaccagcctgacgaaaaaattttatcactaagtgatacgcaagagcaggtgtgacggat

SEQ ID NO: 30 - DNA, 72 bp, Artificial Sequence
  chemically synthesized
atccgtcacacctgctcttgcgtatcacttagtgataaaattttttcgtcaggctggtgttggctcccgtat

SEQ ID NO: 31 - DNA, 71 bp, Artificial Sequence
  chemically synthesized
atacgggagccaacaccagacctacttcagaaacggaaatgttcttagccgtcagagcaggtgtgacggat

SEQ ID NO: 32 - DNA, 71 bp, Artificial Sequence
  chemically synthesized
atccgtcacacctgctctgacggctaagaacatttccgtttctgaagtaggtctggtgttggctcccgtat

SEQ ID NO: 33 - DNA, 72 bp, Artificial Sequence
  chemically synthesized
atacgggagccaacaccaggccaacgaaactcctactacatataatgcttatgcagagcaggtgtgacggat

SEQ ID NO: 34 - DNA, 72 bp, Artificial Sequence
  chemically synthesized
atccgtcacacctgctctgcataagcattatatgtagtaggagtttcgttggcctggtgttggctcccgtat

SEQ ID NO: 35 - DNA, 72 bp, Artificial Sequence; identical to SEQ ID NO: 27
  chemically synthesized

SEQ ID NO: 36 - DNA, 72 bp, Artificial Sequence
  chemically synthesized
atccgtcacacctgctctcgggatcggtaacagcaaaaattagaccagttaggatggtgttggctcccgtat

SEQ ID NO: 37 - DNA, 60 bp, Artificial Sequence
  chemically synthesized
catccgtcacacctgctctggccactaacatggggaccaggtggtgttggctcccgtatc

SEQ ID NO: 38 - DNA, 60 bp, Artificial Sequence
  chemically synthesized
catccgtcacacctgctctgctatcacatgcctgctgaagtggtgttggctcccgtatca

SEQ ID NO: 39 - DNA, 60 bp, Artificial Sequence
  chemically synthesized
catccgtcacacctgctctggggatgtgtggtgttggctcccgtatcaagggcgaattct

SEQ ID NO: 40 - DNA, 61 bp, Artificial Sequence
  chemically synthesized
catccgtcacacctgctctgatcagggaagacgccaacacgtggtgttggctcccgtatca

SEQ ID NO: 41 - DNA, 61 bp, Artificial Sequence
  chemically synthesized
catccgtcacacctgctctgggtggtgttggctcccgtatcaagggcgaattctgcagata

SEQ ID NO: 42 - DNA, 62 bp, Artificial Sequence
  chemically synthesized
catccgtcacacctgctctggggagggtggcgcccgtctcggtggtgttggctcccgtatca

SEQ ID NO: 43 - DNA, 62 bp, Artificial Sequence
  chemically synthesized
catccgtcacacctgctctgggatagggtctcgtgctagatgtggtgttggctcccgtatca

SEQ ID NO: 44 - DNA, 62 bp, Artificial Sequence
  chemically synthesized
catccgtcacacctgctctggaccggcgcttattcctgcttgtggtgttggctcccgtatca

SEQ ID NO: 45 - DNA, 62 bp, Artificial Sequence
  chemically synthesized
catccgtcacacctgcyctggagctgatattggatggtccggtggtgttggctcccgtatca

SEQ ID NO: 46 - DNA, 62 bp, Artificial Sequence
  chemically synthesized
catccgtcacacctgcycygcccagagcaggtgtgacggatgtggtgttggctcccgtatca

SEQ ID NO: 47 - DNA, 62 bp, Artificial Sequence
  chemically synthesized
catccgtcacacctgcycygccggaccatccaatatcagctgtggtgttggctcccgtatca

SEQ ID NO: 48 - DNA, 72 bp, Artificial Sequence
  chemically synthesized
atacgggagccaacaccattaaatcaattgtgccgtgttggtcttgtctcatcgagagcaggtgtgacggat

SEQ ID NO: 49 - DNA, 72 bp, Artificial Sequence
  chemically synthesized
atccgtcacacctgctctcgatgagacaagaccaacacggcacaattgatttaatggtgttggctcccgtat

SEQ ID NO: 50 - DNA, 72 bp, Artificial Sequence
  chemically synthesized
atacgggagccaacaccatttttattatcggtatgatcctacgagttcctcccaagagcaggtgtgacggat

SEQ ID NO: 51 - DNA, 72 bp, Artificial Sequence
  chemically synthesized
atccgtcacacctgctcttgggaggaactcgtaggatcataccgataataaaaatggtgttggctcccgtat

SEQ ID NO: 52 - DNA, 72 bp, Artificial Sequence
  chemically synthesized
atacgggagccaacaccaccgtatatcttattatgcacagcatcacgaaagtgcagagcaggtgtgacggat

SEQ ID NO: 53 - DNA, 72 bp, Artificial Sequence
  chemically synthesized
atccgtcacacctgctctgcactttcgtgatgctgtgcataataagatatacggtggtgttggctcccgtat

SEQ ID NO: 54 - DNA, 72 bp, Artificial Sequence
  chemically synthesized
atacgggagccaacaccattaacgttaagcggcctcacttcttttaatcctttcagagcaggtgtgacggat

SEQ ID NO: 55 - DNA, 72 bp, Artificial Sequence
  chemically synthesized
atccgtcacacctgctctgaaaggattaaaagaagtgaggccgcttaacgttaatggtgttggctcccgtat

SEQ ID NO: 56 - DNA, 72 bp, Artificial Sequence
  chemically synthesized
atccgtcacacctgctctaatatagaggtattgctcttggacaaggtacagggatggtgttggctcccgtat

SEQ ID NO: 57 - DNA, 72 bp, Artificial Sequence
  chemically synthesized
atacgggagccaacaccatccctgtaccttgtccaagagcaatacctctatattagagcaggtgtgacggat

SEQ ID NO: 58 - DNA, 72 bp, Artificial Sequence; identical to SEQ ID NO: 54
  chemically synthesized

SEQ ID NO: 59 - DNA, 72 bp, Artificial Sequence; identical to SEQ ID NO: 55
  chemically synthesized

SEQ ID NO: 60 - DNA, 72 bp, Artificial Sequence
  chemically synthesized
atccgtcacacctgctctaattaggatacggggcaacagaacgagaggggggaatggtgttggctcccgtat

SEQ ID NO: 61 - DNA, 71 bp, Artificial Sequence
  chemically synthesized
atccgtcacacctgctctcggaccaggtcagacaagcacatcggatatccggctggtgttggctcccgtat

SEQ ID NO: 62 - DNA, 72 bp, Artificial Sequence; identical to SEQ ID NO: 60
  chemically synthesized

SEQ ID NO: 63 - DNA, 72 bp, Artificial Sequence
  chemically synthesized
atccgtcacacctgctcttgagtcaaagagtttagggaggagctaacataacagtggtgttggctcccgtat

SEQ ID NO: 64 - DNA, 72 bp, Artificial Sequence
  chemically synthesized
atccgtcacacctgctctaacaacaatgcatcagcgggctgggaacgcatgcggtggtgttggctcccgtat

SEQ ID NO: 65 - DNA, 72 bp, Artificial Sequence
  chemically synthesized
atccgtcacacctgctctgaacaggttataagcaggagtgatagtttcaggatctggtgttggctcccgtat

SEQ ID NO: 66 - DNA, 71 bp, Artificial Sequence
  chemically synthesized
atccgtcacacctgctctcggcggctcgcaaaccgagtggtcagcacccgggttggtgttggctcccgtat

SEQ ID NO: 67 - DNA, 72 bp, Artificial Sequence
  chemically synthesized
atccgtcacacctgctctgcgcaagacgtaatccacaagaccgtgaaaacatagtggtgttggctcccgtat

SEQ ID NO: 68 - DNA, 72 bp, Artificial Sequence
  chemically synthesized
atacgggagccaacaccattcccccctctcgttctgttgccccgtatcctaattagagcaggtgtgacggat

SEQ ID NO: 69 - DNA, 71 bp, Artificial Sequence
  chemically synthesized
atacgggagccaacaccagccggatatccgatgtgcttgtctgacctggtccgagagcaggtgtgacggat

SEQ ID NO: 70 - DNA, 72 bp, Artificial Sequence; identical to SEQ ID NO: 68
  chemically synthesized

SEQ ID NO: 71 - DNA, 72 bp, Artificial Sequence
  chemically synthesized
atacgggagccaacaccactgttatgttagctcctccctaaactctttgactcaagagcaggtgtgacggat

SEQ ID NO: 72 - DNA, 72 bp, Artificial Sequence
  chemically synthesized
atacgggagccaacaccaccgcatgcgttcccagcccgctgatgcattgttgttagagcaggtgtgacggat

SEQ ID NO: 73 - DNA, 72 bp, Artificial Sequence
  chemically synthesized
atacgggagccaacaccagatcctgaaactatcactcctgcttataacctgttcagagcaggtgtgacggat

SEQ ID NO: 74 - DNA, 71 bp, Artificial Sequence
  chemically synthesized
atacgggagccaacaccaacccgggtgctgaccactcggtttgcgagccgccgagagcaggtgtgacggat

SEQ ID NO: 75 - DNA, 72 bp, Artificial Sequence
  chemically synthesized
atacgggagccaacaccactatgttttcacggtcttgtggattacgtcttgcgcagagcaggtgtgacggat

SEQ ID NO: 76 - DNA, 72 bp, Artificial Sequence
  chemically synthesized
atccgtcacacctgctctaggcgtagtgactaagtcgcgcgaaaatcacagcattggtgttggctcccgtat

SEQ ID NO: 77 - DNA, 72 bp, Artificial Sequence
  chemically synthesized
atccgtcacacctgctctcagcggcagctatacagtgagaacggactagtgcgttggtgttggctcccgtat

SEQ ID NO: 78 - DNA, 72 bp, Artificial Sequence
  chemically synthesized
atccgtcacacctgctctggcaaataatactagcgatgatggatctggatagactggtgttggctcccgtat

SEQ ID NO: 79 - DNA, 72 bp, Artificial Sequence
  chemically synthesized
atccgtcacacctgctctgggggtgcgacttagggtaagtgggaaagacgatgctggtgttggctcccgtat

SEQ ID NO: 80 - DNA, 72 bp, Artificial Sequence
  chemically synthesized
atccgtcacacctgctctcaagaggagatgaaccaatcttagtccgacaggcggtggtgttggctcccgtat

SEQ ID NO: 81 - DNA, 72 bp, Artificial Sequence
  chemically synthesized
atccgtcacacctgctctggcccggaattgtcatgacgtcacctacacctcctgtggtgttggctcccgtat

SEQ ID NO: 82 - DNA, 72 bp, Artificial Sequence
  chemically synthesized
atacgggagccaacaccaatgctgtgattttcgcgcgacttagtcactacgcctagagcaggtgtgacggat

SEQ ID NO: 83 - DNA, 72 bp, Artificial Sequence
  chemically synthesized
atacgggagccaacaccaacgcactagtccgttctcactgtatagctgccgctgagagcaggtgtgacggat

SEQ ID NO: 84 - DNA, 72 bp, Artificial Sequence
  chemically synthesized
atacgggagccaacaccagtctatccagatccatcatcgctagtattatttgccagagcaggtgtgacggat

SEQ ID NO: 85 - DNA, 72 bp, Artificial Sequence
  chemically synthesized
atacgggagccaacaccagcatcgtctttcccacttaccctaagtcgcacccccagagcaggtgtgacggat

SEQ ID NO: 86 - DNA, 72 bp, Artificial Sequence
  chemically synthesized
atacgggagccaacaccaccgcctgtcggactaagattggttcatctcctcttgagagcaggtgtgacggat

SEQ ID NO: 87 - DNA, 72 bp, Artificial Sequence
  chemically synthesized
atacgggagccaacaccacaggaggtgtaggtgacgtcatgacaattccgggccagagcaggtgtgacggat

SEQ ID NO: 88 - DNA, 61 bp, Artificial Sequence
  chemically synthesized
gatacgggagccaacaccacccgtatcgttcccaatgcactcagagcaggtgtgacggatg

SEQ ID NO: 89 - DNA, 61 bp, Artificial Sequence
  chemically synthesized
catccgtcacacctgctctgagtgcattgggaacgatacgggtggtgttggctcccgtatg

SEQ ID NO: 90 - DNA, 60 bp, Artificial Sequence
  chemically synthesized
gatacgggagccaacaccacgttcccatacaagttactgacagagcaggtgtgacggatg

SEQ ID NO: 91 - DNA, 60 bp, Artificial Sequence
  chemically synthesized
catccgtcacacctgctctgtcagtaacttgtatgggaacgtggtgttggctcccgtatc

SEQ ID NO: 92 - DNA, 72 bp, Artificial Sequence
  chemically synthesized
atccgtcacccctgctctcgtcgctatgaagtaacaaagataggagcaatcgggtggtgttggctcccgtat

SEQ ID NO: 93 - DNA, 72 bp, Artificial Sequence
  chemically synthesized
atccgtcacacctgctctaacgaagactgaaaccaaagcagtgacagtgctgaatggtgttggctcccgtat

SEQ ID NO: 94 - DNA, 72 bp, Artificial Sequence
  chemically synthesized
atccgtcacacctgctctcggtgacaatagctcgatcagcccaaagtcgtcagatggtgttggctcccgtat

SEQ ID NO: 95 - DNA, 71 bp, Artificial Sequence
  chemically synthesized
atccgtcacacctgctctaacgaaatagaccacaaatcgatactttatgttattggtgttggctcccgtat

SEQ ID NO: 96 - DNA, 72 bp, Artificial Sequence
  chemically synthesized
atccgtcacacctgctctgtcgaatgctctgcctggaagagttgttagcagggatggtgttggctcccgtat

SEQ ID NO: 97 - DNA, 72 bp, Artificial Sequence
  chemically synthesized
atccgtcacacctgctcttaagccgaggggtaaatctaggacaggggtccatgatggtgttggctcccgtat

SEQ ID NO: 98 - DNA, 72 bp, Artificial Sequence
  chemically synthesized
atccgtcacacctgctctactggccggctcagcatgactaagaaggaagttatgtggtgttggctcccgtat

SEQ ID NO: 99 - DNA, 72 bp, Artificial Sequence
  chemically synthesized
atccgtcacacctgctctggtacgaatcacaggggatgctggaagcttggctcttggtgttggctcccgtat

SEQ ID NO: 100 - DNA, 72 bp, Artificial Sequence
  chemically synthesized
atacgggagccaacaccacccgattgctcctatctttgttacttcatagcgacgagagcaggggtgacggat

SEQ ID NO: 101 - DNA, 72 bp, Artificial Sequence
  chemically synthesized
atacgggagccaacaccattcagcactgtcactgctttggtttcagtcttcgttagagcaggtgtgacggat

SEQ ID NO: 102 - DNA, 72 bp, Artificial Sequence
  chemically synthesized
atacgggagccaacaccatctgacgactttgggctgatcgagctattgtcaccgagagcaggtgtgacggat

SEQ ID NO: 103 - DNA, 71 bp, Artificial Sequence
  chemically synthesized
atacgggagccaacaccaataacataaagtatcgatttgtggtctatttcgttagagcaggtgtgacggat

SEQ ID NO: 104 - DNA, 72 bp, Artificial Sequence
  chemically synthesized
atacgggagccaacaccatccctgctaacaactcttccaggcagagcattcgacagagcaggtgtgacggat

SEQ ID NO: 105 - DNA, 72 bp, Artificial Sequence
  chemically synthesized
atacgggagccaacaccatcatggacccctgtcctagatttacccctcggcttaagagcaggtgtgacggat

SEQ ID NO: 106 - DNA, 72 bp, Artificial Sequence
  chemically synthesized
atacgggagccaacaccacataacttccttcttagtcatgctgagccggccagtagagcaggtgtgacggat

SEQ ID NO: 107 - DNA, 72 bp, Artificial Sequence
  chemically synthesized
atacgggagccaacaccaagagccaagcttccagcatcccctgtgattcgtaccagagcaggtgtgacggat

SEQ ID NO: 108 - DNA, 72 bp, Artificial Sequence
  chemically synthesized
atacgggagccaacaccattaaatcaattgtgccgtgttcctcttgtctcatcgagagcaggttgtacggat

SEQ ID NO: 109 - DNA, 72 bp, Artificial Sequence
  chemically synthesized
atccgtacaacctgctctcgatgagacaagaggaacacggcacaattgatttaatggtgttggctcccgtat

SEQ ID NO: 110 - DNA, 72 bp, Artificial Sequence
  chemically synthesized
atacgggagccaacaccagcagtcaagaagttaagagaaaaacaattgtgtataagagcaggtgtgacggat

SEQ ID NO: 111 - DNA, 72 bp, Artificial Sequence
  chemically synthesized
atccgtcacacctgctcttatacacaattgtttttctcttaacttcttgactgctggtgttggctcccgtat

SEQ ID NO: 112 - DNA, 71 bp, Artificial Sequence
  chemically synthesized
atccgtcacacctgctctgcgccacaagattgcggaaagacacccggggggcttggtgttggctcccgtat

SEQ ID NO: 113 - DNA, 71 bp, Artificial Sequence
  chemically synthesized
atacgggagccaacaccaagccccccgggtgtctttccgcaatcttgtggcgcagagcaggtgtgacggat

SEQ ID NO: 114 - DNA, 57 bp, Artificial Sequence
  chemically synthesized
atccgtcacacctgctctggccttatgtaaagcgttgggtggtgttggctcccgtat

SEQ ID NO: 115 - DNA, 57 bp, Artificial Sequence
  chemically synthesized
atacgggagccaacaccacccaacgctttacataaggccagagcaggtgtgacggat

SEQ ID NO: 116 - DNA, 60 bp, Artificial Sequence
  chemically synthesized
catccgtcacacctgctctggttcgccccggtcaaggagagtggtgttggctcccgtatc

SEQ ID NO: 117 - DNA, 60 bp, Artificial Sequence
  chemically synthesized
gatacgggagccaacaccactctccttgaccggggcgaaccagagcaggtgtgacggatg

SEQ ID NO: 118 - DNA, 61 bp, Artificial Sequence
  chemically synthesized
catccgtcacacctgctctggataagatcagcaacaagttagtggtgttggctcccgtatc

SEQ ID NO: 119 - DNA, 60 bp, Artificial Sequence
  chemically synthesized
gatacgggagccaacaccactaacttgttgctgatcttatcagagcaggtgtgacggatg

SEQ ID NO: 120 - DNA, 72 bp, Artificial Sequence
  chemically synthesized
atccgtcacacctgctctccgcacgtaggaccactttggtacacgctcccgtagtggtgttggctcccgtat

SEQ ID NO: 121 - DNA, 72 bp, Artificial Sequence
  chemically synthesized
atccgtcacacctgctctacggatgaacgaagattttaaagtcaagctaatgcatggtgttggctcccgtat

SEQ ID NO: 122 - DNA, 72 bp, Artificial Sequence
  chemically synthesized
atccgtcacacctgctctgtagtgaagagtccgcagtccacgctgttcaactcatggtgttggctcccgtat

SEQ ID NO: 123 - DNA, 72 bp, Artificial Sequence
  chemically synthesized
atccgtcacacctgctctaccggctggcacggttatgtgtgacgggcgaagatatggtgttggctcccgtat

SEQ ID NO: 124 - DNA, 72 bp, Artificial Sequence; identical to SEQ ID NO: 123
  chemically synthesized

SEQ ID NO: 125 - DNA, 58 bp, Artificial Sequence
  chemically synthesized
atccgtcacacctgctctgcgtgtggagcgcctaggtgagtggtgttggctcccgtat

SEQ ID NO: 126 - DNA, 72 bp, Artificial Sequence
  chemically synthesized
atccgtcacacctgctctgatgtccctttgaagagttccatgacgctggctccttggtgttggctcccgtat

SEQ ID NO: 127 - DNA, 72 bp, Artificial Sequence
  chemically synthesized
atacgggagccaacaccactacgggagcgtgtaccaaagtggtcctacgtgcggagagcaggtgtgacggat

SEQ ID NO: 128 - DNA, 72 bp, Artificial Sequence
  chemically synthesized
atacgggagccaacaccatgcattagcttgactttaaaatcttcgttcatccgtagagcaggtgtgacggat

SEQ ID NO: 129 - DNA, 72 bp, Artificial Sequence
  chemically synthesized
atacgggagccaacaccatgagttgaacagcgtggactgcggactcttcactacagagcaggtgtgacggat

SEQ ID NO: 130 - DNA, 72 bp, Artificial Sequence
  chemically synthesized
atacgggagccaacaccatatcttcgcccgtcacacataaccgtgccagccggtagagcaggtgtgacggat

SEQ ID NO: 131 - DNA, 72 bp, Artificial Sequence; identical to SEQ ID NO: 130
  chemically synthesized

SEQ ID NO: 132 - DNA, 58 bp, Artificial Sequence
  chemically synthesized
atacgggagccaacaccactcacctaggcgctccacacgcagagcaggtgtgacggat

SEQ ID NO: 133 - DNA, 72 bp, Artificial Sequence
  chemically synthesized
atacgggagccaacaccaaggagccagcgtcatggaactcttcaaagggacatcagagcaggtgtgacggat

SEQ ID NO: 134 - DNA, 69 bp, Artificial Sequence
  chemically synthesized
atacgggagccaacaccatagtgttgggccaatacggtaacgtgtccttggagagcaggtgtgacggat

SEQ ID NO: 135 - DNA, 72 bp, Artificial Sequence
  chemically synthesized
atccgtcacacctgctctccaaggacacgttaccgacgaattggcccaacactatggtgttggctcccgtat

SEQ ID NO: 136 - DNA, 72 bp, Artificial Sequence
  chemically synthesized
atacgggagccaacaccacacatacgagttatctcgagtagagcatgttttgccagagcaggtgtgacggat

SEQ ID NO: 137 - DNA, 72 bp, Artificial Sequence
  chemically synthesized
atccgtcacacctgctctggcaaaacatgctctactcgagataactcgtatgtgtggtgttggctcccgtat

SEQ ID NO: 138 - DNA, 72 bp, Artificial Sequence
  chemically synthesized
atacgggagccaacaccaggccatctattgttcgtttttctatttatctcacccagagcaggtgtgacggat

SEQ ID NO: 139 - DNA, 72 bp, Artificial Sequence
  chemically synthesized
atccgtcacacctgctctgggtgagataaatagaaaaacgaacaatagatggcctggtgttggctcccgtat

SEQ ID NO: 140 - DNA, 72 bp, Artificial Sequence; identical to SEQ ID NO: 136
  chemically synthesized

SEQ ID NO: 141 - DNA, 72 bp, Artificial Sequence; identical to SEQ ID NO: 137
  chemically synthesized

SEQ ID NO: 142 - DNA, 72 bp, Artificial Sequence
  chemically synthesized
atacgggagccaacaccatccatagctcatctataccctcttccgagtcccaccagagcaggtgtgacggat

SEQ ID NO: 143 - DNA, 72 bp, Artificial Sequence
  chemically synthesized
atccgtcacacctgctctggtgggactcggaagagggtatagatgagctatggatggtgttggctcccgtat

SEQ ID NO: 144 - DNA, 65 bp, Artificial Sequence
  chemically synthesized
atacgggagccaacaccagagcaggtgtgacggatagtgacggatgcagagcaggtgtgacggat

SEQ ID NO: 145 - DNA, 65 bp, Artificial Sequence
  chemically synthesized
atccgtcacacctgctctgcatccgtcactatccgtcacacctgctctggtgttggctcccgtat

SEQ ID NO: 146 - DNA, 72 bp, Artificial Sequence
  chemically synthesized
atacgggagccaacaccaccttatgacgcctcagtaccacatcgtttagtctgtagagcaggtgtgacggat

SEQ ID NO: 147 - DNA, 72 bp, Artificial Sequence
  chemically synthesized
atccgtcacacctgctctacagactaaacgatgtggtactgaggcgtcataaggtggtgttggctcccgtat

SEQ ID NO: 148 - DNA, 72 bp, Artificial Sequence
  chemically synthesized
atacgggagccaacaccacccgtttttgatctaatgaggatacaatattcgtctagagcaggtgtgacggat

SEQ ID NO: 149 - DNA, 72 bp, Artificial Sequence
  chemicall synthesized
atccgtcacacctgctctagacgaatattgtatcctcattagatcaaaaacgggtggtgttggctcccgtat

SEQ ID NO: 150 - DNA, 72 bp, Artificial Sequence
  chemically synthesized
atacgggagccaacaccatcgagctccttggccccgttaggattaacgtgatgtagagcaggtgtgacggat

SEQ ID NO: 151 - DNA, 72 bp, Artificial Sequence
  chemically synthesized
atccgtcacacctgctctacatcacgttaatcctaacggggccaaggagctcgatggtgttggctcccgtat

SEQ ID NO: 152 - DNA, 72 bp, Artificial Sequence
  chemically synthesized
atacgggagccaacaccatcagaaccaaatatacatcttcctatgatatggtggagagcaggtgtgacggat

SEQ ID NO: 153 - DNA, 72 bp, Artificial Sequence
  chemically synthesized
atccgtcacacctgctctccaccatatcataggaagatgtatatttggttctgatggtgttggctcccgtat

SEQ ID NO: 154 - DNA, 72 bp, Artificial Sequence
  chemically synthesized
atacgggagccaacaccacacgattgctcctctcattgttacttcatagcgacgagagcaggtgtgacggat

SEQ ID NO: 155 - DNA, 72 bp, Artificial Sequence
  chemically synthesized
atccgtcacacctgctctcgtcgctatgaagtaacaatgagaggagcaatcgtgtggtgttggctcccgtat

SEQ ID NO: 156 - DNA, 72 bp, Artificial Sequence
  chemically synthesized
gatacgggacgacaccacactatgggtcgtttagcatcaaggctagccaagccagcagaggtgtggtgaatg

SEQ ID NO: 157 - DNA, 72 bp, Artificial Sequence
  chemically synthesized
cattcaccacacctctgctggcttggctagccttgatgctaaacgacccatagtgtggtgtcgtcccgtatc

SEQ ID NO: 158 - DNA, 72 bp, Artificial Sequence
  chemically synthesized
cattcaccacacctctgctggaggaggaagtggtctggagttacttgacatagtgtggtgtcgtcccgtatc

SEQ ID NO: 159 - DNA, 72 bp, Artificial Sequence
  chemically synthesized
gatacgggacgacaccacactatgtcaagtaactccagaccacttcctcctccagcagaggtgtggtgaatg

SEQ ID NO: 160 - DNA, 72 bp, Artificial Sequence
  chemically synthesized
cattcaccacacctctgctggacggaaacaatccccgggtacgagaatcagggtgtggtgtcgtcccgtatc

SEQ ID NO: 161 - DNA, 72 bp, Artificial Sequence
  chemically synthesized
gatacgggacgacaccacaccctgattctcgtacccggggattgtttccgtccagcagaggtgtggtgaatg

SEQ ID NO: 162 - DNA, 72 bp, Artificial Sequence
  chemically synthesized
cattcaccacacctctgctggaaacctaccattaatgagacatgatgcggtggtgtggtgtcgtcccgtatc

SEQ ID NO: 163 - DNA, 72 bp, Artificial Sequence
  chemically synthesized
gatacgggacgacaccacaccaccgcatcatgtctcattaatggtaggtttccagcagaggtgtggtgaatg

SEQ ID NO: 164 - DNA, 72 bp, Artificial Sequence
  chemically synthesized
atccgtcacacctgctctggtggaatggactaagctagctagcgttttaaaaggtggtgttggctcccgtat

SEQ ID NO: 165 - DNA, 72 bp, Artificial Sequence
  chemically synthesized
atccgtcacacctgctctgtaaggggggggaatcgctttcgtcttaagatgacatggtgttggctcccgtat

SEQ ID NO: 166 - DNA, 59 bp, Artificial Sequence
  chemically synthesized
atccgtcacacctgctctgccggaccatccaatatcagctgtggtgttggctcccgtat

SEQ ID NO: 167 - DNA, 71 bp, Artificial Sequence
  chemically synthesized
atccgtcacacctgctctatccgtcacgcctgctctatccgtcacacctgctctggtgttggctcccgtat

SEQ ID NO: 168 - DNA, 72 bp, Artificial Sequence
  chemically synthesized
atccgtcacacctgctctatcaaatgtgcagatatcaagacgatttgtacaagatggtgttggctcccgtat

SEQ ID NO: 169 - DNA, 72 bp, Artificial Sequence
  chemically synthesized
atccgtcacacctgctctgtagatggcaaggcataagcgtccggaacgatagaatggtgttggctcccgtat

SEQ ID NO: 170 - DNA, 72 bp, Artificial Sequence; identical to SEQ ID NO: 169
  chemically synthesized

SEQ ID NO: 171 - DNA, 72 bp, Artificial Sequence
  chemically synthesized
atacgggagccaacaccaccttttaaaacgctagctagcttagtccattccaccagagcaggtgtgacggat

SEQ ID NO: 172 - DNA, 72 bp, Artificial Sequence
  chemically synthesized
atacgggagccaacaccatgtcatcttaagacgaaagcgattccccccccttacagagcaggtgtgacggat

SEQ ID NO: 173 - DNA, 59 bp, Artificial Sequence
  chemically synthesized
atacgggagccaacaccacagctgatattggatggtccggcagagcaggtgtgacggat

SEQ ID NO: 174 - DNA, 71 bp, Artificial Sequence
  chemically synthesized
atacgggagccaacaccagagcaggtgtgacggatagagcaggcgtgacggatagagcaggtgtgacggat

SEQ ID NO: 175 - DNA, 72 bp, Artificial Sequence
  chemically synthesized
atacgggagccaacaccatcttgtacaaatcgtcttgatatctgcacatttgatagagcaggtgtgacggat

SEQ ID NO: 176 - DNA, 72 bp, Artificial Sequence
  chemically synthesized
atacgggagccaacaccattctatcgttccggacgcttatgccttgccatctacagagcaggtgtgacggat

SEQ ID NO: 177 - DNA, 72 bp, Artificial Sequence; identical to SEQ ID NO: 176
  chemically synthesized

SEQ ID NO: 178 - DNA, 59 bp, Artificial Sequence; identical to SEQ ID NO: 166
  chemically synthesized

SEQ ID NO: 179 - DNA, 72 bp, Artificial Sequence; identical to SEQ ID NO: 164
  chemically synthesized

SEQ ID NO: 180 - DNA, 72 bp, Artificial Sequence
  chemically synthesized
atccgtcacacctgctcttaaagtagaggctgttctccagacgtcgcaggaggatggtgttggctcccgtat

SEQ ID NO: 181 - DNA, 72 bp, Artificial Sequence; identical to SEQ ID NO: 169
  chemically synthesized

SEQ ID NO: 182 - DNA, 72 bp, Artificial Sequence; identical to SEQ ID NO: 169
  chemically synthesized

SEQ ID NO: 183 - DNA, 59 bp, Artificial Sequence; identical to SEQ ID NO: 173
  chemically synthesized

SEQ ID NO: 184 - DNA, 72 bp, Artificial Sequence
  chemically synthesized
atccgtcacacctgctcttgggcaggagcgagagactctaatggtaagcaagaatggtgttggctcccgtat

SEQ ID NO: 185 - DNA, 72 bp, Artificial Sequence
  chemically synthesized
atccgtcacacctgctctccaacaaggcgaccgaccgcatgcagatagccaggttggtgttggctcccgtat

SEQ ID NO: 186 - DNA, 59 bp, Artificial Sequence; identical to SEQ ID NO: 173
  chemically synthesized

SEQ ID NO: 187 - DNA, 72 bp, Artificial Sequence; identical to SEQ ID NO: 171
  chemically synthesized

SEQ ID NO: 188 - DNA, 72 bp, Artificial Sequence
  chemically synthesized
atacgggagccaacaccatcctcctgcgacgtctggagaacagcctctactttaagagcaggtgtgacggat

SEQ ID NO: 189 - DNA, 72 bp, Artificial Sequence; identical to SEQ ID NO: 176
  chemically synthesized

SEQ ID NO: 190 - DNA, 72 bp, Artificial Sequence; identical to SEQ ID NO: 176
  chemically synthesized

SEQ ID NO: 191 - DNA, 59 bp, Artificial Sequence; identical to SEQ ID NO: 166
  chemically synthesized

SEQ ID NO: 192 - DNA, 72 bp, Artificial Sequence
  chemically synthesized
atacgggagccaacaccattcttgcttaccattagagtctctcgctcctgcccaagagcaggtgtgacggat

SEQ ID NO: 193 - DNA, 72 bp, Artificial Sequence
  chemically synthesized
atacgggagccaacaccaacctggctatctgcatgcggtcggtcgccttgttggagagcaggtgtgacggat

SEQ ID NO: 194 - DNA, 72 bp, Artificial Sequence; identical to SEQ ID NO: 169
  chemically synthesized

SEQ ID NO: 195 - DNA, 72 bp, Artificial Sequence
  chemically synthesized
atccgtcacacctgctctaaccaaaagggtaggagaccaagctagcgatttggatggtgttggctcccgtat

SEQ ID NO: 196 - DNA, 59 bp, Artificial Sequence; identical to SEQ ID NO: 166
  chemically synthesized

SEQ ID NO: 197 - DNA, 72 bp, Artificial Sequence
  chemically synthesized
atccgtcacacctgctctgaagcctaacggagaagatggccctactgccgtaggtggtgttggctcccgtat

SEQ ID NO: 198 - DNA, 72 bp, Artificial Sequence
  chemically synthesized
atccgtcacacctgctctactaaacaagggcaaactgtaaacacagtaggggcgtggtgttggctcccgtat

SEQ ID NO: 199 - DNA, 68 bp, Artificial Sequence
  chemically synthesized
atccgtcacacctgctctggtgttggctcccgtatagcttggctcccgtatggtgttggctcccgtat

SEQ ID NO: 200 - DNA, 72 bp, Artificial Sequence
  chemically synthesized
atccgtcacacctgctctgtcgcgatgatgagcagcagcgcaggagggagggggtggtgttggctcccgtat

SEQ ID NO: 201 - DNA, 57 bp, Artificial Sequence
  chemically synthesized
atccgtcacacctgctctgatcagggaagacgccaacactggtgttggctcccgtat

SEQ ID NO: 202 - DNA, 72 bp, Artificial Sequence; identical to SEQ ID NO: 176
  chemically synthesized

SEQ ID NO: 203 - DNA, 72 bp, Artificial Sequence
  chemically synthesized
atacgggagccaacaccatccaaatcgctagcttggtctcctacccttttggttagagcaggtgtgacggat

SEQ ID NO: 204 - DNA, 59 bp, Artificial Sequence; identical to SEQ ID NO: 173
  chemically synthesized

SEQ ID NO: 205 - DNA, 72 bp, Artificial Sequence
  chemically synthesized
atacgggagccaacaccacctacggcagtagggccatcttctccgttaggcttcagagcaggtgtgacggat

SEQ ID NO: 206 - DNA, 72 bp, Artificial Sequence
  chemically synthesized
atacgggagccaacaccacgcccctactgtgtttacagtttgcccttgtttagtagagcaggtgtgacggat

SEQ ID NO: 207 - DNA, 68 bp, Artificial Sequence
  chemically synthesized
atacgggagccaacaccatacgggagccaagctatacgggagccaacaccagagcaggtgtgacggat

SEQ ID NO: 208 - DNA, 72 bp, Artificial Sequence
  chemically synthesized
atacgggagccaacaccaccccctccctcctgcgctgctgctcatcatcgcgacagagcaggtgtgacggat

SEQ ID NO: 209 - DNA, 57 bp, Artificial Sequence
  chemically synthesized
atacgggagccaacaccagtgttggcgtcttccctgatcagagcaggtgtgacggat

SEQ ID NO: 210 - DNA, 58 bp, Artificial Sequence
  chemically synthesized
atccgtcacacctgctctgtccaaaggctacgcgttaacgtggtgttggctcccgtat

SEQ ID NO: 211 - DNA, 58 bp, Artificial Sequence
  chemically synthesized
atccgtcacacctgctctggagcaatatggtggagaaacgtggtgttggctcccgtat

SEQ ID NO: 212 - DNA, 59 bp, Artificial Sequence; identical to SEQ ID NO: 166
  chemically synthesized

SEQ ID NO: 213 - DNA, 72 bp, Artificial Sequence
  chemically synthesized
atccgtcacacctgctctgaacaggatagggattagcgagtcaactaagcagcatggtgttggctcccgtat

SEQ ID NO: 214 - DNA, 72 bp, Artificial Sequence
  chemically synthesized
atccgtcacacctgctctggcggacaggaaataagaatgaacgcaaaatttatctggtgttggctcccgtat

SEQ ID NO: 215 - DNA, 72 bp, Artificial Sequence
  chemically synthesized
atccgtcacacctgctctacgcaacgcgacaggaacattcattatagaatgtgttggtgttggctcccgtat

SEQ ID NO: 216 - DNA, 72 bp, Artificial Sequence
  chemically synthesized
atccgtcacacctgctctcggctgcaatgcgggagagtaggggggaaccaaacctggtgttggctcccgtat

SEQ ID NO: 217 - DNA, 72 bp, Artificial Sequence
  chemically synthesized
atccgtcacacctgctctatgactggaacacgggtatcgatgattagatgtccttggtgttggctcccgtat

SEQ ID NO: 218 - DNA, 58 bp, Artificial Sequence
  chemically synthesized
atacgggagccaacaccacgttaacgcgtagcctttggacagagcaggtgtgacggat

SEQ ID NO: 219 - DNA, 58 bp, Artificial Sequence
  chemically synthesized
atacgggagccaacaccacgtttctccaccatattgctccagagcaggtgtgacggat

SEQ ID NO: 220 - DNA, 59 bp, Artificial Sequence; identical to SEQ ID NO: 173
  chemically synthesized

SEQ ID NO: 221 - DNA, 72 bp, Artificial Sequence
  chemically synthesized
atacgggagccaacaccatgctgcttagttgactcgctaatccctatcctgttcagagcaggtgtgacggat

SEQ ID NO: 222 - DNA, 72 bp, Artificial Sequence
  chemically synthesized
atacgggagccaacaccagataaattttgcgttcattcttatttcctgtccgccagagcaggtgtgacggat

SEQ ID NO: 223 - DNA, 72 bp, Artificial Sequence
  chemically synthesized
atacgggagccaacaccaacacattctataatgaatgttcctgtcgcgttgcgtagagcaggtgtgacggat

SEQ ID NO: 224 - DNA, 72 bp, Artificial Sequence
  chemically synthesized
atacgggagccaacaccaggtttggttcccccctactctcccgcattgcagccgagagcaggtgtgacggat

SEQ ID NO: 225 - DNA, 72 bp, Artificial Sequence
  chemically synthesized
atacgggagccaacaccaaggacatctaatcatcgatacccgtgttccagtcatagagcaggtgtgacggat